(19) United States
(12) Patent Application Publication
(10) Pub. No.: US 2019/0262425 A1
(43) Pub. Date: Aug. 29, 2019

(54) USES OF IL-41

(71) Applicant: The Regents of the University of California, Oakland, CA (US)

(72) Inventors: Albert ZLOTNIK, Oakland, CA (US); Irina USHACH, San Bruno, CA (US)

(73) Assignee: The Regents of the University of California, Oakland, CA (US)

(21) Appl. No.: 16/308,078

(22) PCT Filed: Jun. 10, 2017
(86) PCT No.: PCT/US2017/036921

§ 371 Date: Dec. 7, 2018

Related U.S. Application Data
(60) Provisional application No. 62/348,506, filed on Jun. 10, 2016.

(51) Int. Cl. A61K 38/20; C07K 14/54; C07K 16/24; A61P 19/02; A61P 29/00; A61P 31/04

(52) U.S. Cl. CPC A61K 38/20; C07K 14/54; A61P 31/04; A61P 19/02; A61P 29/00; C07K 16/24

(57) ABSTRACT

Methods of using Metrnl/IL-41 to identify inflammation, inflammatory and autoimmune diseases, infection and cancer in a subject are provided. The methods include determining the serum or plasma levels of Metrnl/IL-41 in the subject. Methods of modulating a condition selected from cytokine release syndrome, systemic immune response syndrome, cytokine storm, or a combination thereof, using Metrnl/IL-41 or antibodies against Metrnl/I-41 are also provided.

### CROSS-REFERENCE TO RELATED APPLICATIONS

This application claims the benefit of U.S. Provisional Patent Application No. 62/348,506, filed on Jun. 10, 2016, which is incorporated by reference herein.

### STATEMENT REGARDING FEDERALLY SPONSORED RESEARCH OR DEVELOPMENT

This invention was made with Government support under Grant No. AI117556 from the National Institutes of Health. The Government has certain rights in this invention.

### BACKGROUND

### Field of the Invention

The invention relates to uses of Interleukin-41.

### Related Art

There are many inflammatory/autoimmune diseases where inflammation plays a critical role. Recently it has become clear that inflammatory process can also contribute significantly to the progression of cancer. The problem is that not all of the molecules involved in the inflammatory process are known. In addition, there is an ongoing need for better diagnostic and prognostic agents for inflammatory and autoimmune conditions.

### SUMMARY

This application describes the use of Meteorin-like (Metrnl)/IL-41 as a diagnostic/prognostic biomarker for inflammatory/autoimmune diseases and cancer, and also identifies the meteorin-like-meteorin-like receptor interaction as a target for the development of monoclonal antibodies for use in many autoimmune/inflammatory/infectious diseases and cancer (the inventors previously referred to Metrnl as “IL-39”, but now refer to it here as “IL-41” because recently a different cytokine has been called IL-39 in the literature [Wang et al. 2016, A novel cytokine IL-23p19/Ebi3 (IL-39) mediates inflammation in lupus-like mice, Eur. J. Immunol. 46(6) 1343-50]; also, the inventors have identified another novel cytokine that they have called IL-40 [U.S. patent application Ser. No. 15/036,207, filed on May 12, 2016]). Meteorin-like/IL-41 can also be used directly for therapeutic purposes in vivo. Thus, embodiments relate to:

i) A biomarker for prognostic/diagnostic uses in autoimmune/inflammatory diseases.

ii) A biomarker for prognostic/diagnostic uses in cancers.

iii) Meteorin-like/IL-41 as a therapeutic for inflammatory/autoimmune diseases and cancer.

iv) Antibodies that inhibit the binding of Meteorin-like/IL-41 to it cognate receptor as therapeutics for inflammatory/autoimmune disease and cancer.

v) Methods for identification of the Meteorin-like/IL-41 receptor.

In one aspect, a method of modulating inflammation in a subject in need thereof is provided. The method includes administering to the subject an effective amount of Metrnl/IL-41, a bioactive fragment of Metrnl/IL-41, a genetically or chemically modified Metrnl/IL-41, or any combination thereof, or administering one or more antibodies against Metrnl/IL-41.

In a further aspect, a method of modulating a condition selected from cytokine release syndrome (CRS), systemic immune response syndrome (SIRS), cytokine storm, or a combination thereof, in a subject in need of such modulating, is provided. The method includes administering to the subject: an effective amount of Meteorin-like (Metrnl/IL-41), a bioactive fragment of Metrnl/IL-41, a genetically or chemically modified Metrnl/IL-41, or a combination thereof; or administering one or more anti-Metrnl/IL-41 antibodies.

In some embodiments: a) the modulating comprises reducing the severity of the condition; b) the subject can have sepsis; c) an effective amount of Meteorin-like (Metrnl/IL-41), a bioactive fragment of Metrnl/IL-41, a genetically or chemically modified Metrnl/IL-41, or a combination thereof, is administered; d) an effective amount of one or more anti-Metrnl/IL-41 antibodies is administered, which can be Metrnl/IL-41 neutralizing antibodies; e) after the administering, the subject is monitored for one or more symptoms of the condition; f) in some embodiments, the symptoms that are monitored include serum levels of interleukin-6, interleukin-1, or TNFα, or a combination thereof; g) the route of administration can be intravenously; or h) any combination of a)-g).

In some embodiments, the cytokine storm may be a result of one or more conditions or diseases that the subject has, including, but not limited to, bacteremia, bacterial sepsis, cellulitis, cholecystitis, community-acquired pneumonia, diabetic foot infection, erysipelas, HIV (acute retroviral syndrome), infective endocarditis, influenza, intra-abdominal infections, meningitis, necrotizing fasciitis, nosocomial pneumonia, pelvic inflammatory disease, prostatitis, pseudomembranous colitis (Clostridium difficile), pyelonephritis, septic arthritis, or systemic inflammatory response syndrome (SIRS), toxic shock syndrome, urinary tract infection, viral syndrome, acute mesenteric ischemia, acute respiratory distress syndrome, alcohol withdrawal, burns, cirrhosis, connective tissue disease, deep venous thrombosis, dehydration, DKA, drug overdose, drug reaction, electrical injuries, erythema multiforme, gastrointestinal bleeding, gout, graft versus host disease, hemorrhagic shock, intestinal perforation, malignancy, myocardial infarction, pancreatitis, peripheral ischemia, pulmonary embolism, toxic epidermal necrolysis, transfusion reactions or trauma.

In some embodiments, the cytokine release syndrome may be a result of one or more conditions or diseases that the subject has including, but not limited to, sepsis, CAR-T cell toxicity, and diseases caused by superantigens such as, for example, staphylococcal enterotoxin B, toxin shock toxin.

In addition, administration of Metrnl/IL-41, a bioactive fragment of Metrnl/IL-41, a genetically or chemically modified Metrnl/IL-41, or a combination thereof, should also be effective in inflammatory diseases such as autoimmune diseases (for example, rheumatoid arthritis, Crohn's disease, inflammatory bowel disease, and psoriasis).

In another aspect, a method of identifying the inflammatory status of a subject is provided. The method includes determining the serum, plasma, urine, cerebrospinal fluid or bronchoalveolar lavage levels of Metrnl/IL-41 in the subject.

In a further aspect, a method of identifying the presence of an inflammatory or autoimmune disease in a subject is provided. The method includes determining the serum, plasma, urine, cerebrospinal fluid or bronchoalveolar lavage levels of Metrnl/IL-41 in the subject.

In another aspect, a method of identifying the presence of an infection in a subject is provided. The method includes determining the serum, plasma, urine, cerebrospinal fluid or bronchoalveolar lavage levels of Metrnl/IL-41 in the subject.

In a further aspect, a method of identifying a subset of inflammatory or autoimmune diseases in a subject is provided. The method includes determining the serum, plasma, urine, cerebrospinal fluid or bronchoalveolar lavage levels of Metrnl/IL-41 in the subject.

In another aspect, a method of identifying the course of an inflammatory or autoimmune disease in a subject is provided. The method includes determining the serum, plasma, urine, cerebrospinal fluid or bronchoalveolar lavage levels of Metrnl/IL-41 in the subject.

In a further aspect, a method of diagnosing cancer in a subject is provided. The method includes determining the serum, plasma, urine, cerebrospinal fluid or bronchoalveolar lavage levels of Metrnl/IL-41 in the subject.

In another aspect, a method of treating a cytokine storm in a subject in need thereof is provided. The method includes administering an effective amount of an antibody against Metrnl/IL-41 to the subject.

In a further aspect, a method of treating rheumatoid arthritis, juvenile idiopathic arthritis, psoriasis (arthritic or plaque), ankylosing spondylitis, Crohn's disease, ulcerative colitis, hidradenitis suppurativa, pelvic inflammatory disease, or asthma in a subject in need thereof is provided. The method includes administering an effective amount of Metrnl/IL-41 to the subject.

In another aspect, a method of identifying the Metrnl/IL-41 receptor in a cell expressing the receptor is provided. The method includes binding labeled Metrnl/IL-41 to membrane proteins of the cell. In some embodiments, the labeled Metrnl/IL-41 is bound to the candidate receptor protein(s) and the complex is isolated by, e.g., immunoprecipitation, for further analysis of the receptor proteins by, e.g., mass spectroscopy.

### BRIEF DESCRIPTION OF THE DRAWINGS

For a more complete understanding of the present invention, reference is now made to the following descriptions taken in conjunction with the accompanying drawings, in which:

FIG. 1 is a Clustal Omega alignment of the amino acid sequences of Meteorin (NP_076947.1; SEQ ID NO:1) and Meteorin-like (NP_001004431.1, SEQ ID NO:2).

FIG. 2 is a panel showing that Metrnl/IL-41 is highly expressed in human activated monocytes. Mean expression values with error bars (standard deviation) plotted against tissue/cell type (x axis). The highest expression values correspond to activated monocytes (LPS, 30 hr) or activated peripheral blood mononuclear cells (PBMC, PMA+ionomycin, 30 hrs)). Other sites of significant expression include oral mucosa, skin, trachea and esophagus, other mucosal and skin-associated samples and trigeminal and dorsal root ganglia of the nervous system.

FIG. 3 is a panel of graphs showing Metrnl/IL-41 production, as measured by qPCR or ELISA, is regulated by cytokines in mouse peritoneal macrophages. (3A) Induction (qPCR) as compared to resting macrophages: of Metrnl/IL-41 by IL-4 and IFNγ; of CXCL10 by IL-4, IFNγ and Metrnl/IL-41; of Arginase I by IL-4, IFNγ and Metrnl/IL-41. (3B) Induction (ELISA) over time by Metrnl/IL-41 by IL-4, IL-13, IFNγ and LPS. (3C) Induction (qPCR): of Metrnl/IL-41 by GM-CSF and M-CSF; of TNFα by GM-CSF and M-CSF; and, of IL-10 by GM-CSF and M-CSF. (D) Induction (ELISA) of Metrnl/IL-41 by GM-CSF, M-CSF, M-CSF+IL-4, and M-CSF+IFNγ.

FIG. 4 is a panel showing changes in Metrnl/IL-41 expression. (4A, 4B) Induction of Metrnl/IL-41 expression (ELISA) in macrophages by TNFα, IL-17a, IL-12, IL-4, and IL-1β, and reduction of Metrnl/IL-41 expression by IFNγ and TGFβ. (4C, 4D) Induction of Metrnl/IL-41 (ELISA) in serum and peritoneal exudate fluid by i.p. thioglycollate injection-induced inflammatory response.

FIG. 5 is a panel of graphs showing the induction of IL-10, IL-6, CCL5, CCL17, CXCL1, CCL2 and CCL20 (ELISA) in peritoneal macrophages by Metrnl/IL-41.

FIG. 6 is a panel of graphs showing the induction of IL-6 and IL-10 in bone marrow macrophages by Metrnl/IL-41.

FIG. 7 is a graph showing that Metrnl/IL-41 blocks LPS-induced IL-6 production by macrophages. Three separate preparations of Metrnl/IL-41 were tested.

FIG. 8 is a panel showing the strategy used to generate an Il-41−/− mouse. Embryonic Stem (ES) cells were used containing a selection cassette that inserted into the mouse Metrnl locus by homologous recombination (8A). These ES clones were microinjected into C57BL/6 blastocysts, which were transferred into pseudo-pregnant females. Resulting chimeras were crossed with WT C57BL/6 mice and the pups were screened for germline transmission. Il-41Neo/+ heterozygotes were subsequently bred with FLPeR mice to remove the neomycin resistance cassette (8B). The Metrnl/IL-41 Neo−/loxP+ mice were then bred to CRE mice to delete the loxP-flanked target region (8C). Finally, heterozygote Il-41+/− mice were intercrossed to successfully generate Il-41−/− mice. The deletion of the target region was verified by PCR (8D) and Metrnl/IL-41 production by bone marrow macrophages (ELISA) (8E).

FIG. 9 is a panel of images of inflamed uterus (left image) and inflamed kidney (right image) obtained from Metrnl/IL-41−/− knock-out mice and control wild type (WT) mice.

FIG. 10 is a panel showing examples of altered levels of protein RNA levels in the inflamed uterus from the Metrnl/IL-41−/− mice as compared to levels in the WT mice.

FIG. 11 is a panel showing examples of altered levels of protein RNA levels in the inflamed kidney from the Metrnl/IL-41−/− mice as compared to levels in the WT mice.

FIG. 12 is a panel showing flow cytometric analysis of Siglec F and Ly6G expression on CD11b+ immune cells obtained from various tissue compartments of WT and Metrnl/IL-41−/− mice.

FIG. 13 is a panel of graphs showing the frequency of neutrophils (top) and eosinophils (bottom) in blood, bone marrow and spleen compartments of WT and Metrnl/IL-41−/− mice. The Metrnl/IL-41−/− mice exhibited significant neutrophilia.

FIG. 14 is a graph showing that Metrnl/IL-41−/− macrophages activated by LPS produce more G-CSF as compared to WT macrophages.

FIG. 15 is a graph showing the reduction in the frequency of plasmacytoid dendritic cells (pDCs) in the spleens of Metrnl/IL-41−/− mice.

FIG. 16 is a panel of graphs showing the reduction in CD4+ and CD8+ cells in the spleens of Metrnl/IL-41−/− mice.

FIG. 17 is a panel of graphs showing altered total serum IgG levels in Metrnl/IL-41−/− mice compared to WT mice. Total IgG levels reflect significantly lower levels of IgG2b and IgG3 in IL-41−/− mice.

FIG. 18 is a graph showing that activated T cell production of an early phase inflammatory chemokine (CCL3) is eliminated in the Metrnl/IL-41−/− mice.

FIG. 19 is a graph showing that activated T cell production of an inflammatory cytokine (IFNγ) is significantly reduced in the Metrnl/IL-41−/− mice.

FIG. 20 is a panel of graphs showing that activated T cells from Metrnl/IL-41−/− mice produce significantly altered levels of many cytokines as compared to WT control mice. For each cytokine measured lane 1 is WT cells at rest, lane 2 is IL-41−/− cells at rest, lane 3 is WT cells stimulated by α-CD3/α-CD28 antibodies, and lane 4 is IL-41−/− cells stimulated by α-CD3/α-CD28 antibodies.

FIG. 21 is a panel of graphs showing that activated T cells from Metrnl/IL-41−/− mice produce significantly altered levels of many chemokines as compared to WT control mice. For each chemokine measured lane 1 is WT cells at rest, lane 2 is IL-41−/− cells at rest, lane 3 is WT cells stimulated by α-CD3/α-CD28 antibodies, and lane 4 is IL-41−/− cells stimulated by α-CD3/α-CD28 antibodies.

FIG. 22 is a panel showing that either of two strains of Toxoplasma gondii cause increased Metrnl/IL-41 production in the THP-1 monocytic cell line.

FIG. 23 is a panel of graphs showing significant upregulation of Metrnl/IL-41 in serum or in peritoneal exudate occurs for at least 14 days in WT mice infected by Toxoplasma gondii.

FIG. 24 is a panel showing that Metrnl/IL-41−/− mice show significantly reduced Toxoplasma gondii burden (24A, 24B) and significantly increased survival (24C) after exposure to the intracellular parasite. Cytokines and chemokine responses were reduced in the Metrnl/IL-41−/− mice (24D).

FIG. 25 is a graph showing that fibroblasts from human skin express elevated Metrnl/IL-41 (left) and the expression of Metrnl/IL-41 by skin keratinocytes is increased by activation with IFNγ.

FIG. 26 is a panel of graphs showing that METRNL/IL-41 is strongly over-expressed in human psoriatic skin.

FIG. 27 is a graph showing that METRNL/IL-41 is significantly upregulated in synovial membranes of human rheumatoid arthritis.

### DETAILED DESCRIPTION

In aspects and embodiments of the invention, the inflammation can be, but is not limited to, whole system inflammation and cytokine storm such as occurs in cytokine response syndrome (CRS), sepsis, systemic inflammatory response syndrome (SIRS), hyperinflammatory syndrome, cardiovascular inflammation such as occurs in atherosclerosis, inflammation of the skin, joints, pericardium, vena cava, aorta, thyroid, eye (conjunctiva), oral cavity, nasal sinus, pharynx, esophagus, stomach, duodenum, jejunum, ileum, colon, anus, trachea, bronchus, alveoli, ear, kidney, urethra, bladder, ovary, fallopian tube, uterus, cervix, vagina, vulva, penis, or testes.

In aspects and embodiments of the invention, the inflammatory or autoimmune disease can be, but is not limited to, psoriasis, rheumatoid arthritis, ankylosing spondylitis, Crohn's disease, ulcerative colitis, hidradenitis suppurativa, pelvic inflammatory disease, or asthma. Other conditions include inflammatory bowel disease (IBD), irritable bowel syndrome (IBS), or lung inflammations such as chronic asthma, allergy and idiopathic pulmonary fibrosis (IPF). The identified subset of inflammatory or autoimmune diseases can be psoriasis (arthritic or plaque), juvenile rheumatoid or juvenile idiopathic arthritis.

In aspects and embodiments of the invention, the identified infection can be, but is not limited to, a gram (+) bacterial infection, gram (−) bacterial infection, yeast infection, fungal infection or viral infection.

In aspects and embodiments of the invention, the diagnosed cancer can be, but is not limited to, glioblastoma, lymphoma, leukemia, myeloma, carcinoma, adenocarcinoma, sarcoma, or mesothelioma. The cancer can be blood born/hematopoietic (e.g., lymphoma, leukemia, and the like) or solid tumor (e.g., lung, colorectal, breast, ovarian, glioblastoma, and the like).

Standard methods for producing and making the ligands, receptors, and variants can be applied. Standard recombinant methods can be developed, including design of recombinant nucleic acids encoding constructs. See, e.g., Thompson D. A. Cell and Molecular Biology Manual 2011. Expression vectors, e.g., with promoters operably linked to coding regions, can be devised. Cells comprising the vectors are provided, including both prokaryote cells and eukaryote cells. Compatible expression methodologies can also be developed. The nucleotide and amino acid sequences of Metrnl/IL-41 can be accessed at Genbank mRNA (NM_001004431.2) or protein (AAI18639.1), which are incorporated by reference herein.

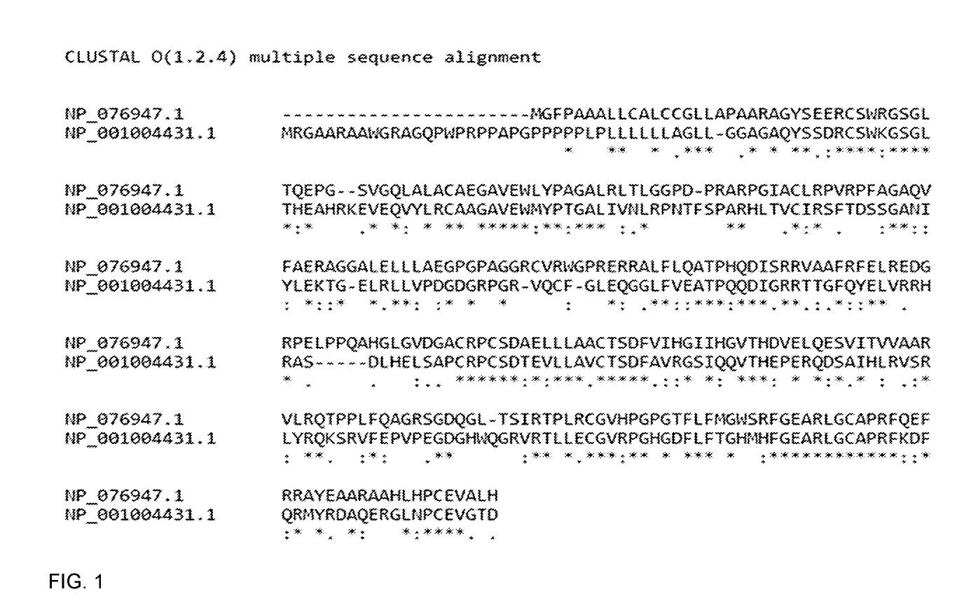

An amino acid alignment of using the Clustal Omega alignment program is provided for Meteorin and Meteorin-like polypeptides in FIG. 1.

Typically, a polynucleotide that encodes the polypeptide of interest is placed under the control of a promoter that is functional in the desired host cell. An extremely wide variety of promoters is well known, and can be used in expression vectors of embodiments of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, embodiments the invention provide expression cassettes into which the nucleic acids that encode the relevant functional polypeptides are incorporated for high level expression in a desired host cell (see, e.g., Ream W and Field K. G. Molecular Biology Techniques. Academic Press. 2012).

Substantially pure compositions of at least about 70, 75, 80, 85, 90% homogeneity are preferred, and 92, 95, 98 to 99% or more homogeneity are most preferred. The purified polypeptides may also be used, e.g., as immunogens for antibody production, which antibodies may be used in immunoselection purification methods.

For example, several macrophage cell lines (such as RAW 247) cells produce IL-41 when stimulated with lipopolysaccharide (LPS). IL-41 can be purified from these supernatants by immunoprecipitation with a commercially available anti-Metrnl/IL-41 antibody (R&D systems, Minneapolis, Minn., USA; Cat #AF6679). Supernatants can be concentrated by commercially available devices (for example, Amicon Ultra 15 with a 3,000 Molecular weight cutoff), for example by filling with 15 ml of supernatant and centrifuging according to manufacturer's instructions. The concentrated sample can be recovered when the tube reaches the desired volume (i.e., 0.2-1.0 ml). Concentrated supernatants can be incubated with anti-Metrnl antibody and the immunoprecipitate can be purified using commercially available kits based on Protein A binding for example.

In another example, recombinant mouse METRNL/IL-41, amino acids Gln46-Glu311 (Accession #NP_659046.1) with a C-terminal His tag can be expressed in CHO cells. The recombinant protein has a predicted molecular mass of approximately 31.4 kD. The DTT-reduced protein migrates at approximately 37 kD, and non-reduced proteins migrate at approximately 36 kD by SDS-PAGE. The predicted N-terminal amino acid is Gln. Resulting IL-41 is purified with Ni-NTA and sizing columns. Purity of the eluted protein can be judged by Coomassie Blue SDS PAGE gel staining. Sterile material can be prepared by 0.22 μm filtering of the concentrated protein solution, and the material can be diluted in PBS, pH 7.2.

A polypeptide, peptide, or antibody may be modified at the protein level. Included within the scope of the invention are polypeptides, peptides, protein fragments or other derivatives or analogs that are differentially modified, for example by glycosylation, acetylation, phosphorylation, amidation, pegylation, derivatization by known protecting/blocking groups, and proteolytic cleavage. Any number of chemical modifications may be carried out by known techniques, including but not limited to specific chemical cleavage by cyanogen bromide, trypsin, chymotrypsin, papain, V8 protease, NaBH₄; acetylation, formylation, pegylation, farnesylation, oxidation, and reduction. For example, polyethylene glycol (PEG) can be added to Meteorin-like/IL-41 or a peptide of Meteorin-like/IL-41 to change the pharmacodynamics of the protein/peptide in vivo (i.e., increase its half-life in vivo) (for pegylation, see Grace M, Youngster S, Gitlin G, Sydor W, Xie L, Westreich L, Jacobs S, Brassard D, Bausch J, Bordens R. Structural and biologic characterization of pegylated recombinant IFN-alpha2b. J Interferon Cytokine Res. 2001 December; 21(12):1103-15, incorporated by reference herein; see Ramon J, Saez V, Baez R, Aldana R, Hardy E. PEGylated interferon-alpha2b: a branched 40K polyethylene glycol derivative. Pharm Res. 2005 August; 22(8):1374-86, incorporated by reference herein).

Active fragments of Meteorin-like/IL-41 can be obtained by producing two parts of IL-41 (amino and carboxy terminal parts) and testing each part for activity in a Meteorin-like/IL-41 biological assay. For example, each part can be tested for stimulation of macrophage cytokine production (e.g., production of IL-6). The amino acid sequence of the human Meteorin-like/IL-41 protein and examples of fragments (shown in single letter amino acid notation) are as follows:

### Human Protein (Signal Sequence Underlined)

(SEQ ID NO. 3)
MRGAARAAWGRAGQPWPRPPAPGPPPPPLPLLLLLLAGLLGGAGAQYSSD
RCSWKGSGLTHEAHRKEVEQVYLRCAAGAVEWMYPTGALIVNLRPNTFSP
ARHLTVCIRSFTDSSGANIYLEKTGELRLLVPDGDGRPGRVQCFGLEQGG
LFVEATPQQDIGRRTTGFQYELVRRHRASDLHELSAPCRPCSDTEVLLAV
CTSDFAVRGSIQQVTHEPERQDSAIHLRVSRLYRQKSRVFEPVPEGDGHW
QGRVRTLLECGVRPGHGDFLFTGHMHFGEARLGCAPRFKDFQRMYRDAQE
RGLNPCEVGTD

### Amino Terminal Fragment:

(SEQ ID NO. 4)
QYSSDRCSWKGSGLTHEAHRKEVEQVYLRCAAGAVEWMYPTGALIVNLRP
NTFSPARHLTVCIRSFTDSSGANIYLEKTGELRLLVPDGDGRPGRVQCFG
LEQGGLFVEATPQQDIGRRTTGEQYELVRRHRA

### Carboxy Terminal Fragment:

(SEQ ID NO. 5)
SDLHELSAPCRPCSDTEVLLAVCTSDFAVRGSIQQVTHEPERQDSAIHLR
VSRLYRQKSRVFEPVPEGDGHWQGRVRTLLECGVRPGHGDFLFTGHMHFG
EARLGCAPRFKDFQRMYRDAQERGLNPCEVGTD.

Different formulations of Metrnl/IL-41 or antibodies against Metrnl/IL-41 can be used (sterile, buffered, slow release, controlled release, stabilizers, ointments, etc.) depending on the optimal route of administration. See, e.g., Niazi S. K. Handbook of Pharmaceutical Manufacturing Formulations Informa Healthcare 2012.

The exact dose of Metrnl/IL-41 or antibodies against Metrnl/IL-41 will depend on the purpose of the treatment, and will be ascertainable by one skilled in the art using known techniques. See, e.g., Ansel, et al., Pharmaceutical Dosage Forms and Drug Delivery; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding; and Pickar (1999) Dosage Calculations. As is known in the art, adjustments for protein degradation, systemic versus localized delivery, and rate of new protease synthesis, as well as the age, body weight, general health, sex, diet, time of administration, drug interaction, and the severity of the condition may be necessary, and will be ascertainable with some experimentation by those skilled in the art.

An effective amount, including a therapeutically effective amount, of a protein, protein fragment, small molecule, drug or antibody, is an amount that promotes or enhances the well-being of the subject with respect to the medical treatment of his/her condition. A list of nonexhaustive examples of this includes extension of the subject's life by any period of time, a decrease in pain to the subject that can be attributed to the subject's condition, a decrease in the severity of the disease or condition, including a decrease in the severity of cytokine release syndrome, systemic immune response syndrome, and cytokine storm, or an improvement in the prognosis of the condition or disease.

Various pharmaceutically acceptable excipients are well known in the art. As used herein, “pharmaceutically acceptable excipient” includes a material which, when combined with an active ingredient of a composition, allows the ingredient to retain biological activity and without causing disruptive reactions with the subject's immune system. Such may include stabilizers, preservatives, salt or sugar complexes or crystals, and the like. See, e.g., Niazi S. K. Handbook of Pharmaceutical Manufacturing Formulations Informa Healthcare 2012.

Exemplary pharmaceutical carriers include sterile aqueous of non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms. Administration routes may include the following: topical, systemic, respiratory, oral, eye, implant, vaginal, anal, suppository, devices with control release, sublingual, buccal, nasal, inhalation, parenteral, intraorgan, subcutaneous, intradermal, intramuscular, intravenous, and the like.

An antibody is an immunologic binding agent such as IgG, IgM, IgA, IgD and IgE. Techniques for preparing and using various antibody-based constructs and fragments are well known in the art. Means for preparing and characterizing antibodies are also well known in the art (See, for example, Harlow and Lane, “Antibodies: A Laboratory Manual,” Cold Spring Harbor Laboratory, 1988, incorporated by reference herein). Monoclonal antibodies (mAbs) are recognized to have certain advantages, e.g., reproducibility and large-scale production. Thus, monoclonal antibodies of the human, murine, monkey, rat, hamster, rabbit and even chicken origin, are contemplated for use. In some embodiments, an antibody-like molecule that has an antigen binding region may be appropriate. Examples of such anti-body like molecules include, but are not limited to, antibody fragments such as Fab′, Fab, F(ab′)₂, single domain antibodies (DABs), Fv, scFv (single chain Fv), and the like.

Polyclonal antibodies can be prepared in a wide range of animal species. Typically, the animal used for production of antisera is a rabbit, a mouse, a rat, a hamster, a guinea pig or a goat. To increase immunogenicity, use of adjuvants and conjugation to a carrier protein such as, but not limited to, keyhole limpet hemocyanin or bovine serum albumin are well known procedures.

A monoclonal antibody can be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain. The immunizing composition is administered in a manner effective to stimulate antibody producing cells.

For example, following several immunizations, the presence of anti-Metrnl/IL-41 antibodies in the serum of the mouse can be assayed by testing the serum by enzyme-linked immunosorbant assay (ELISA). Once the presence of anti-Metrnl/IL-41 antibodies is confirmed in the serum of a given mouse, its spleen can be fused to a myeloma cell suitable for the production of monoclonal antibodies using several techniques like PEG-driven fusion or electrical techniques. The resulting hybridomas can be selected in HAT medium and screened for the production of anti-Metrnl/IL-41 antibodies by ELISA.

A polyclonal or monoclonal antibody can be further purified, if desired, using filtration, centrifugation and various chromatographic methods such as HPLC or affinity chromatography.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions. Thus, humanized antibodies against Metrnl/IL-41 are included in some embodiments, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference herein. Methods for the development of antibodies that are “custom-tailored” to the patient's disease are likewise known and such custom-tailored antibodies are also contemplated.

An antibody against Metrnl/IL-41 should recognize the mature protein, not the signal peptide. Most anti-Metrnl/IL-41 antibodies are expected to be neutralizing because most antibodies against cytokines are expected to neutralize biological activity. Antibodies are very large compared to cytokines, so an antibody will likely affect cytokine binding to its specific receptor via steric effects. Assays for identifying Metrnl/IL-41 neutralizing antibody include measuring the ability of the antibody to block the ability of Metrnl/IL-41 to induce IL6 or CXCL1 production by macrophages, and measuring the ability of Metrnl/IL-41 to inhibit IL-6 production induced by LPS in macrophages.

IL-6 is a prototype pro-inflammatory cytokine, along with TNFα and IL-1β. Together, these cytokines mediate many of the symptoms typical of inflammation and cytokine release syndrome. Furthermore, it has been shown that IL-6 is a critical cytokine that mediates cytokine release syndrome (see Tanyi, J. L., et al., Possible Compartmental Cytokine Release Syndrome in a Patient With Recurrent Ovarian Cancer After Treatment With Mesothelin-targeted CAR-T Cells, J. Immunother. 2017 April; 40(3):104-107, incorporated by reference herein). Also, neutralization of IL-6 prevents many of the negative effects of cytokine release syndrome.

Based on experiments described herein, Metrnl/IL-41 expression and regulation correlates with inflammation. Also, Metrnl/IL-41 is induced in macrophages by LPS, and Metrnl/IL-41 is induced in vivo by thioglycollate, an inflammatory stimulus. In addition, Metrnl/IL-41 will block production of the pro-inflammatory cytokine IL-6 induced by LPS, which is a simple model of a cytokine storm situation. Further, regarding the experiments with T. Gondii, since death in infected mice is due to an intense cytokine storm, the robust survival of knockout Metrnl/IL-41−/− mice compared to WT mice, illustrates the relationship between Metrnl/IL-41 and the complexities of the cytokine storm. The Metrnl/IL-41−/− mice survive because they cannot mount a similarly intense cytokine storm, an event that is known to be the fatal root cause in WT mice. Thus, administering Metrnl/IL-41 to a subject experiencing cytokine release syndrome, systemic immune response syndrome, or cytokine storm, is expected to modulate these conditions, including reducing the severity of the cytokine release syndrome, systemic immune response syndrome, and cytokine storm. Alternatively, administering a neutralizing antibody against Metrnl/IL-41 can also modulate the severity of the cytokine release syndrome, systemic immune response syndrome or cytokine storm. The difference will depend on the timing of the administration of either Metrnl/Il-41 or an antibody against Metrnl/IL-41 from the onset of symptoms in a subject.

Assays for detecting the presence of Metrnl/IL-41 include, but are not limited to, ELISA, polymerase chain reaction (PCR), or fluorescence-activated cell sorting (FACS) assays. Immunodetection methods for detecting Metrnl/IL-41 can include ELISA, radioimmunoassay (RiA), fluoroimmunoassay, chemiluminescent assay, bioluminescent assay, Western blotting, and immunohistochemistry. In these methods, a sample is contacted with a first antibody that has affinity for the target protein to form immune complexes, and then the immune complexes are detected, for example, by a label attached to the first antibody (such as a radioactive, fluorescent or enzyme label), or by means of a secondary binding molecule (such as a second antibody) that has affinity for the first antibody. The secondary molecule can be linked to a label for detection.

qPCR is a popular method for quantitation of mRNA corresponding to specific genes. There are a number of systems that can be used for quantitative PCR, including commercially available systems (Roche Diagnostic, Indianapolis, Ind., USA, LightCycler Systems; Thermofisher, Carlsbad, Calif., USA, ABI Real time PCR; BioRad, Hercules, Calif., USA, Real Time PCR Systems). Each of these systems offers practical solutions for quantitative PCR. They require specific primers that should be readily available for Meteorin-like and/or many cytokines and chemokines that can be measured using these methods.

Fluorescence Activated Cell analysis is a popular method for analyzing the expression of proteins in cells of the immune system. There are a number of instruments designed to do this including commercially available ones (Becton-Dickinson, San Jose, Calif., USA). There are many vendors of monoclonal antibodies conjugated to fluorochromes that can be used to label the cells and analyze them in the FACS instruments (R&D systems; Thermofisher; BioLegend).

There are recently developed methods to measure the concentration of many cytokines and chemokines in a single sample (Legendplex, BioLegend). These kits allow the simultaneous measurement of cytokines and chemokines by flow cytometry (see the World Wide Web at biolegend.com/legendplex).

Serum and plasma levels of Metrnl/IL-41 above normal can be indicative of, for example, cancer, inflammation, autoimmune disease, altered homeostasis, diabetes, obesity, atherosclerosis, thrombosis, or an aneurysm. Serum and plasma levels of Metrnl/IL-41 below normal can be indicative of, for example, anorexia or malnutrition.

Metrnl/IL-41 labeled with a tag (biotin or HIS-tag, for example) can be used to identify the Metrnl/IL-41 receptor in cell lines or in cells from the immune system. Microarrays can be used to identify membrane proteins that are expressed by cells that also express the Metrnl/IL-41 receptor. Candidate IL-41 receptor proteins can be confirmed by expressing them in transfected cells and testing the ability of labeled Metrnl/IL-41 to bind naive or transfected cells.

In aspects and embodiments of the invention, the subject can be, e.g., a mammal, a primate, a human, a farm animal, a companion animal, a human, a poultry species, a cow, a horse, a goat, a cat, a sheep, a rodent, a dog, a pig, a chicken, a duck, a turkey, a quail, or a goose. A display or exhibition animal may also be treated, e.g., zoo or performing animal, including pinipeds, whales, dolphins, lions, tigers, and other veterinary subjects including snakes, frogs or fish.

The present invention may be better understood by referring to the accompanying examples, which are intended for illustration purposes only and should not in any sense be construed as limiting the scope of the invention.

### EXAMPLE 1

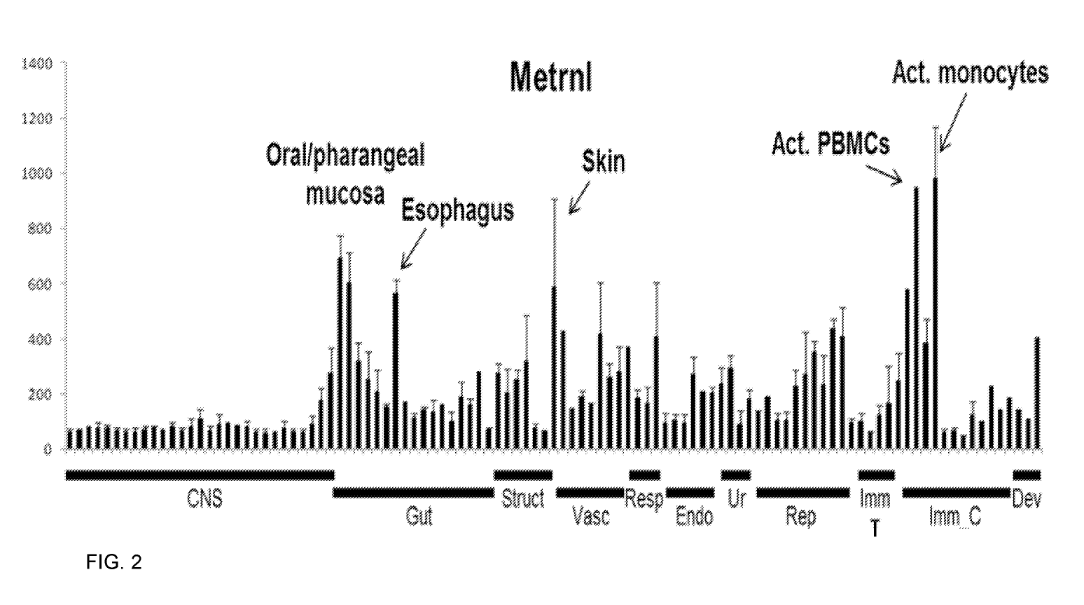

Identification of METRNL/IL-41 expression: The identification of the subset of genes encoding secreted proteins allowed us the opportunity to investigate whether there were novel cytokines that remained to be discovered. We identified a small secreted protein of 311 aminoacids with a 45 aminoacid signal peptide, predicting a mature protein of 266 aminoacids (˜29 KDa) encoded by a gene annotated as “Meteorin-like” (metrnl). Metrnl is related to a known neurotrophic growth factor called meteorin (metrn), which is expressed in the Central Nervous System (CNS). However, as shown in FIG. 2, in contrast to Meteorin, metrnl is not expressed significantly in the CNS, with the exception of expression in dorsal root ganglia and trigeminal ganglia tissues, and is instead expressed in barrier tissues (i.e. skin and mucosa) and, importantly, its highest expression is by activated monocytes (Table I). Mean intensity indicates hybridization signal strength of the Metrnl probeset (225955_at) to cDNA prepared from each of these human tissues. This inducible expression pattern suggested that metrnl represents a novel cytokine (FIG. 2, Table I). The expression pattern in vascular, mucosal and barrier tissues suggests that metrnl may also be produced by epithelial or endothelial cells. This is because it is expressed in tissues that include substantial proportion of epithelial cells (for example mammary gland, oral cavity) and is also expressed in vascular tissues like aorta (Table 1)

TABLE 1
Tope ten sites of METRNL expression in the human
body. Data derived from BIGE database (FIG. 1A).

| Tissue or cell type: | Mean intensity: |
|---|---|
| Monocytes, activated | 981.0 |
| PBMC, activated | 946.6 |
| Oral mucosa | 690.0 |
| Pharyngeal mucosa | 604.0 |
| Skin | 588.7 |
| PBMC | 579.4 |
| Esophagus | 564.4 |
| Nipple (cross section) | 435.8 |
| Pericardium | 428.8 |
| Aorta | 418.9 |

The BIGE database has been described (Gene expression analyses reveal molecular relationships among 20 regions of the human CNS. Roth R B, Hevezi P, Lee J, Willhite D, Lechner S M, Foster A C, Zlotnik A. Neurogenetics. 2006 May; 7(2):67-80; Effects of RNA degradation on gene expression analysis of human postmortem tissues. Lee J, Hever A, Willhite D, Zlotnik A, Hevezi P. FASEB J. 2005 August; 19(10):1356-8, incorporated by reference herein). We have used it to identify novel genes associated with several tissues or cells (P. A. Gerber, P. Hevezi, B. A. Buhren, C. Martinez, H. Schrumpf, M. Gasis, S. Grether-Beck, J. Krutmann, B. Homey, A. Zlotnik, Systematic identification and characterization of novel human skin-associated genes encoding membrane and secreted proteins, PLoS One 8 (2013) e63949, incorporated by reference herein). To construct the BIGE database, samples from 105 different tissues and cell types of the human body were analyzed for gene expression using U133 2.0 genearrays (Affymetrix, Santa Clara, Calif.). The resulting data were normalized as described (Gene expression analyses reveal molecular relationships among 20 regions of the human CNS. Roth R B, Hevezi P, Lee J, Willhite D, Lechner S M, Foster A C, Zlotnik A. Neurogenetics. 2006 May; 7(2):67-80, incorporated by reference herein), and probesets corresponding to METRN (232269_x_at) or METRNL (225955_at) (probeset identification numbers corresponding to Meteorin or Meteorin-like in the Affymetrix U133 gene arrays) were used to determine the expression of the METRN or METRNL-like genes in the human body.

Expression in trigeminal and dorsal root ganglia sites suggest METRNL/IL-41 may play a role in nervous system function through immunomodulatory or pro- or anti-inflammatory effects on, or expression by, barrier cells or glial cells at those sites.

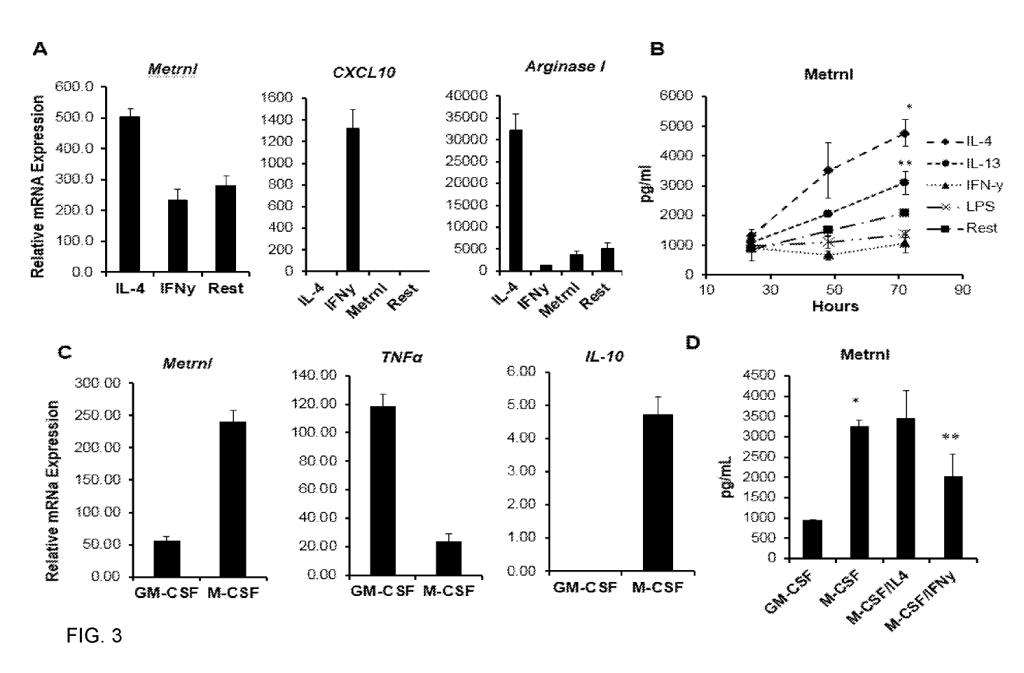

Immunoregulatory cytokines differentially modulate the production of Metrnl/IL-41 by macrophages and other cells. We observed that both macrophages and dendritic cells produce Metrnl/IL-41 upon activation. Using up to date techniques we confirmed that Metrnl/IL-41 is produced by alternatively activated M2 macrophages (FIG. 3). Basal level of IL-41 is about 1000 pg/ml and, with LPS, IL-41 expression is increased. This correlates with inflammatory responses.

For the experimental results described in FIG. 3, (A) peritoneal exudate macrophages from C57BL mice were cultured under either M1 (IFNγ) or M2 (IL-4) polarizing conditions. 8 h post stimulation Metrnl expression was measured by Q-PCR. Metrnl expression was elevated in response to IL-4 and it was suppressed by IFNγ. M1 marker CXCL10 and M2 markers Arginase I were measured in the polarized macrophages to confirm successful polarization. (B) Metrnl protein levels were measured by ELISA in supernatants from macrophages cultured in the presence of IL-4, IL-13, IFNγ and LPS for 24, 48 and 72 h. Production of Metrnl was increased in peritoneal cavity macrophages in response to IL-4 (*P=0.001) and IL-13 (**P=0.02). (C) The Metrnl expression levels were measured by Q-PCR in GM-CSF- or M-CSF-derived BMM (GM-BMM and BMM, respectively). Metrnl was elevated in BMM and paralleled expression of another BMM cytokine (IL-10). (D) Metrnl levels were measured by ELISA in GM-BMM and BMM. Metrnl levels were elevated in supernatants from BMM when compared to GM-BMM (*P=0.0001). Culturing BMM in the presence of IL-4 led to an increase of Metrnl production whereas IFNγ suppressed Metrnl production (**P=0.02).

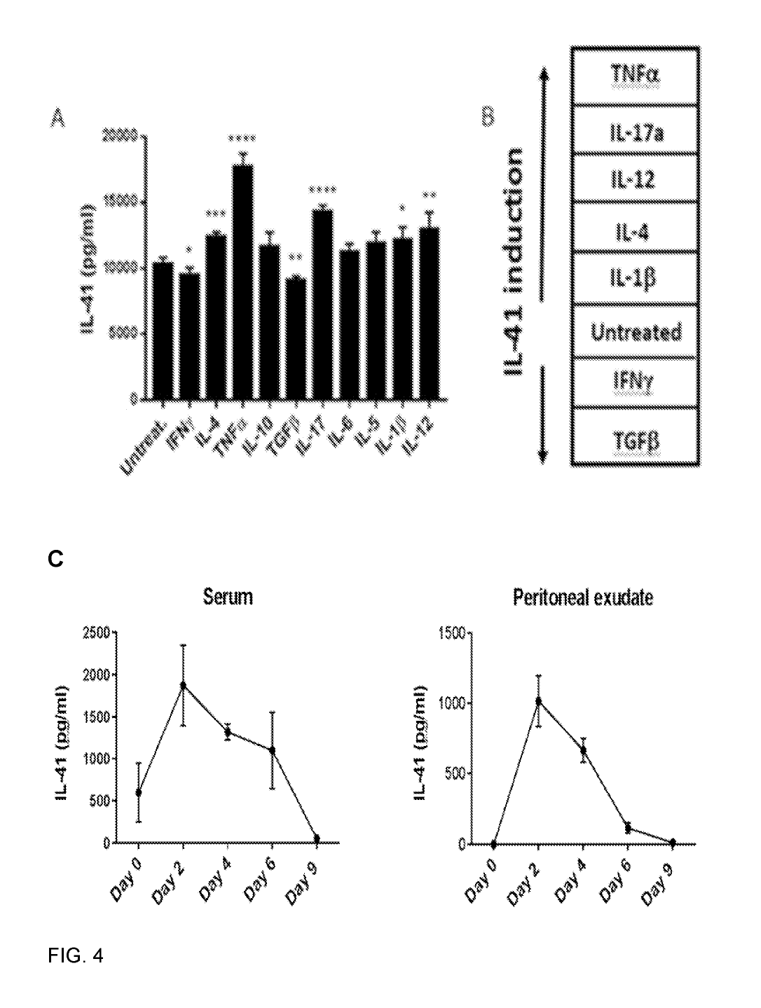

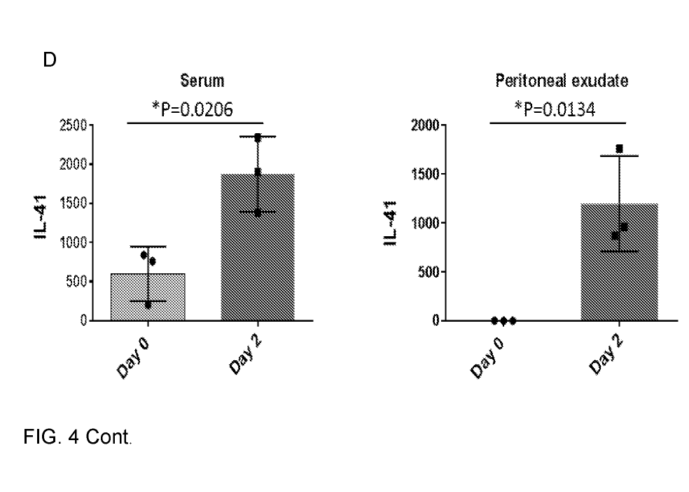

Furthermore, the production of Metrnl/IL-41 is also induced in macrophages by TNFα or IL-17, indicating that it is produced during inflammatory responses. In fact, TNFα, a well-known proinflammatory cytokine is the most powerful inducer of Metrnl/IL-41 in macrophages indicating that Metrnl/IL-41 is normally produced during inflammatory responses (FIGS. 4A and B). In other experiments, the highest expression observed in macrophages is consistently induced by TNFα or IL17, while IFNγ or TGFβ consistently inhibit its expression. This induction within the context of an inflammatory response was confirmed using a thioglycollate inflammation-induction animal model in which the thioglycollate drives recruitment of macrophages to the peritoneal cavity (FIG. 4C, D). Mice were injected intraperitoneally with thioglycolate to induce an inflammatory response within the peritoneum and the serum and peritoneal exudate were tested for Metrnl/IL-41. These data indicate that Metrnl/IL-41 is normally produced during an inflammatory response and is measurable at the site of inflammation and in the circulation.

For the results described in FIG. 4, bone-marrow was isolated from murine femurs and cultured in DMEM with 50 ng/mL M-CSF (BioLegend, San Diego, Calif.). After 3 days, non-adherent cells were removed and fresh medium was added. Bone Marrow Derived Macrophages (BMDM) were used after 7 days in culture. For stimulation, BMDM were cultured in the presence of TNFα, IL-4, IFNγ, TGF_{β}, IL-17A, IL-6, IL-10 or IL-12 (BioLegend). All cytokines were used at 50 ng/ml. Bone marrow derived macrophages (BMDM) were cultured in the presence of various cytokines for 24 hours prior to measurement of IL-41 levels in supernatants by ELISA. (*p<0.05; **p<0.02; ***p<0.01; ****p<0.005. TNFα induced the highest expression of IL-41 in BMDM, but IL-17a, IL12 and IL4 also induced significant increases in IL-41 levels. IFNγ and TGFβ reduced baseline production of IL-41 by BMDM. Levels of IL-41 in serum and peritoneal exudate were measured by ELISA over the course of sterile inflammation caused by thioglycollate injection, and in both cases they were significantly elevated compared to control (uninflamed) mice. Differences in levels of IL-41 in serum or peritoneal exudate from thioglycollate-injected mice were statistically significant at 2 days.

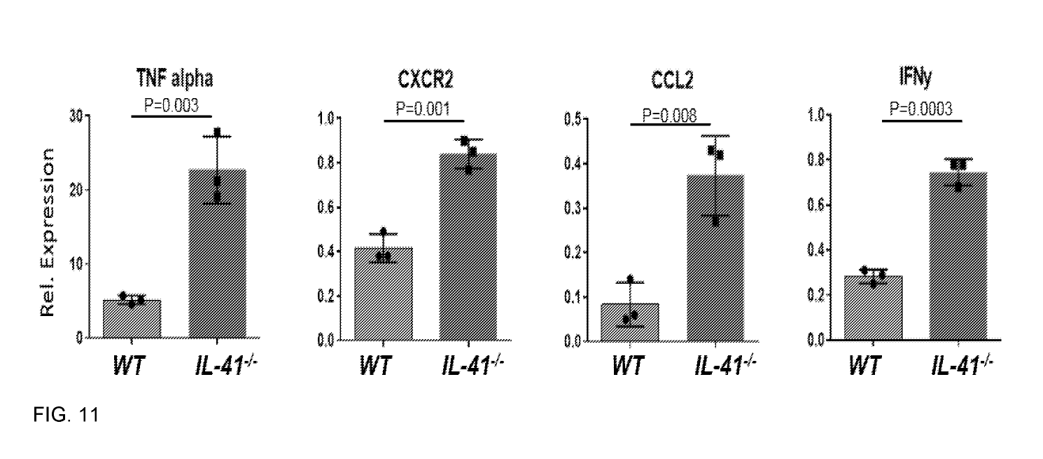

Evidence of statistically significant inhibition of basal expression of Metrnl/IL-41 by IFNγ or by TGFβ is novel (FIG. 4). Evidence of strongest induction of expression of Metrnl/IL-41 by TNFα, IL-17 and IL-12 (FIG. 4), all highly pro-inflammatory cytokines, is also novel, particularly since US2003/0018165 indicated that a combination of TNFα and IFNγ reduced Metrnl/IL-41 expression by peritoneal exudate macrophages (see FIG. 11 of that application). Together these results regarding bidirectional control of expression of Metrnl/IL-41 by pro-inflammatory and anti-inflammatory mediators strongly suggest that Metrnl/IL-41 is involved in inflammatory and fibrotic processes, particularly in light of Metrnl/IL-41's bioactivities described below. These data indicate that Metrnl is going to participate in different immune responses in different ways. For example, it should be reduced or absent in the microenvironment of a developing Th1 response (because IFNγ inhibits production) but elevated in the microenvironment of a developing-late Th17 response (because IL17A induces it). It is also upregulated in inflammatory responses. The novel finding is that it is not ‘always up’ but it can be predicted that its levels will vary depending on the type of immune response. The fact that there are normal levels of Metrnl in plasma suggests that Metrnl may be participating in homoestatic functions of the immune system, for example, by maintaining tolerance.

Metrnl/IL-41 Modulates Production of Cytokines and Chemokines by Macrophages and Other Cells.

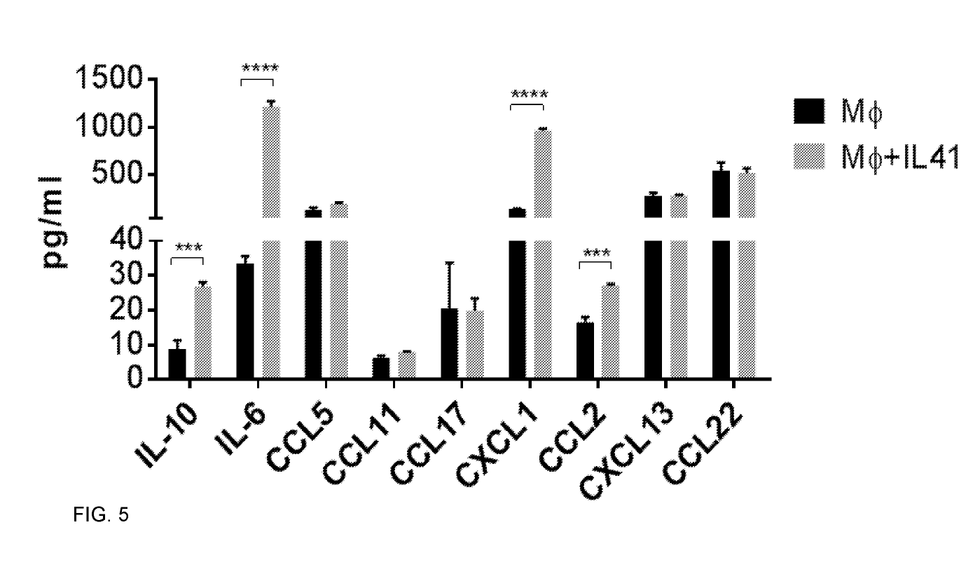

To study the effects of Metrnl/IL-41 on cytokine and chemokine production, peritoneal cavity macrophages from naïve mice were harvested and incubated with or without Metrnl/IL-41 protein in cell culture for 24 hours. Supernatants from the cultures were harvested and tested by ELISA for the levels of many cytokines and chemokines (FIG. 5). The production of IL-10, IL-6, CXCL1 and CCL2 were all induced by Metrnl/IL-41.

For the results described in FIG. 5, peritoneal cavity macrophages were incubated in the presence of IL-41 prior to measuring levels of 26 different cytokines and chemokines in supernatant samples using Legendplex (BioLegend). The expression of IL-10, IL-6, CCL2 and CXCL1 was significantly increased by IL-41. The induction of IL-6 and IL-10 production in bone marrow macrophages by IL-41 was verified by ELISA. Mean+/−SEM. **:p<0.03; ***:p<0.02; ****:p<0.01. Representative experiments (out of two) shown.

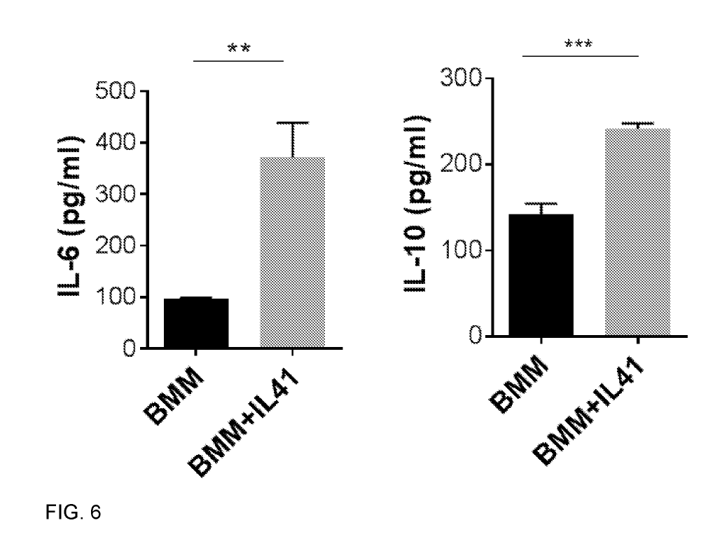

The effects of Metrnl/IL-41 were also studied on cultured mouse bone marrow macrophages, and Metrnl/IL-41 induced both IL-6 and IL-10 production (FIG. 6). These results also indicate that Metrnl/IL-41 is associated with inflammatory processes.

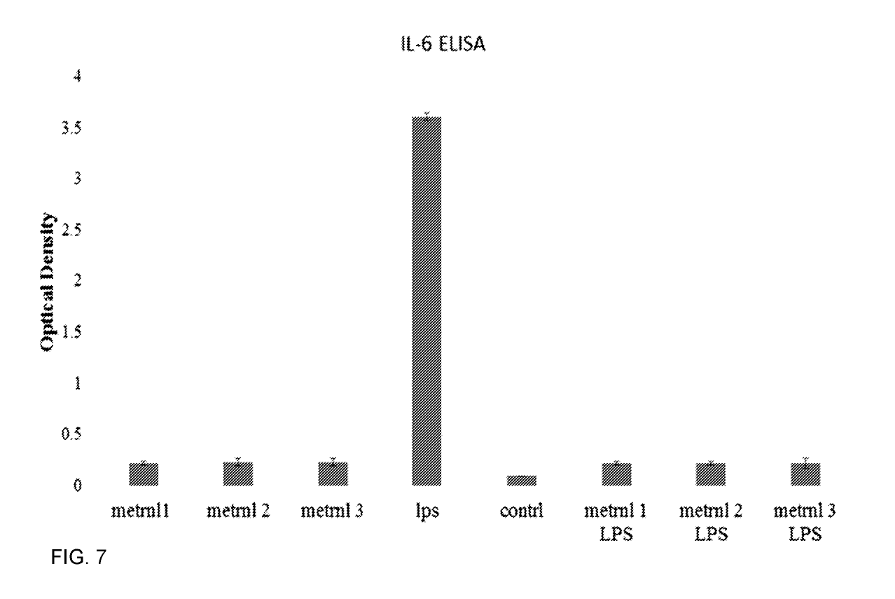

LPS induces inflammatory cytokine (e.g. IL-6) production by macrophages (FIG. 7). For the results described in FIG. 7, 5×105 RAW 264.7 cells (mouse macrophage cell line) were incubated with 200 ng/ml of IL-41 (three different batches) for 48 h and the supernatants were tested for IL-6 levels by ELISA (BioLegend). The addition of LPS (500 ng/ml) induced strong production of IL-6, but the addition of Metrnl/IL-41 ablated the production of IL-6 by the RAW 264.7 cells.

LPS induction of strong pro-inflammatory cytokine release is the root cause of diseases such as sepsis and other diseases associated with a cytokine storm or SIRS. However, in the presence of Metrnl/IL-41 this induction is completely blocked. This shows that Metrnl/IL-41 is a strong modulator of an extreme inflammatory response, in this case a negative modulator of that response.

Metrnl/IL-41 is Produced During Inflammatory Responses and Regulates Cell Properties.

These data indicate that Metrnl/IL-41 is a molecule induced by inflammatory processes and that it may serve an immunoregulatory role. The specific regulation of its production reflects a specific role in different immune responses and perhaps other functions. In summary, the product of the Metrnl gene is a small secreted protein (˜27 kDa) produced by activated macrophages, is present in inflammatory environments, is able to regulate expression of cytokines and chemokines involved in inflammation, and whose production is specifically regulated by several cytokines that play important roles in various immune responses (Th1, Th2, Th17, Tregs). Taken together, these observations indicate the Metrnl gene encodes a novel cytokine that we suggest should be named Interleukin 41 (Metrnl/IL-41). We decided to undertake the biological characterization of Metrnl/IL-41. However, because it is a poorly characterized gene, there were no biological tools available to study Metrnl/IL-41. We therefore decided to produce a Metrnl/Il-41−^{/-} mouse.

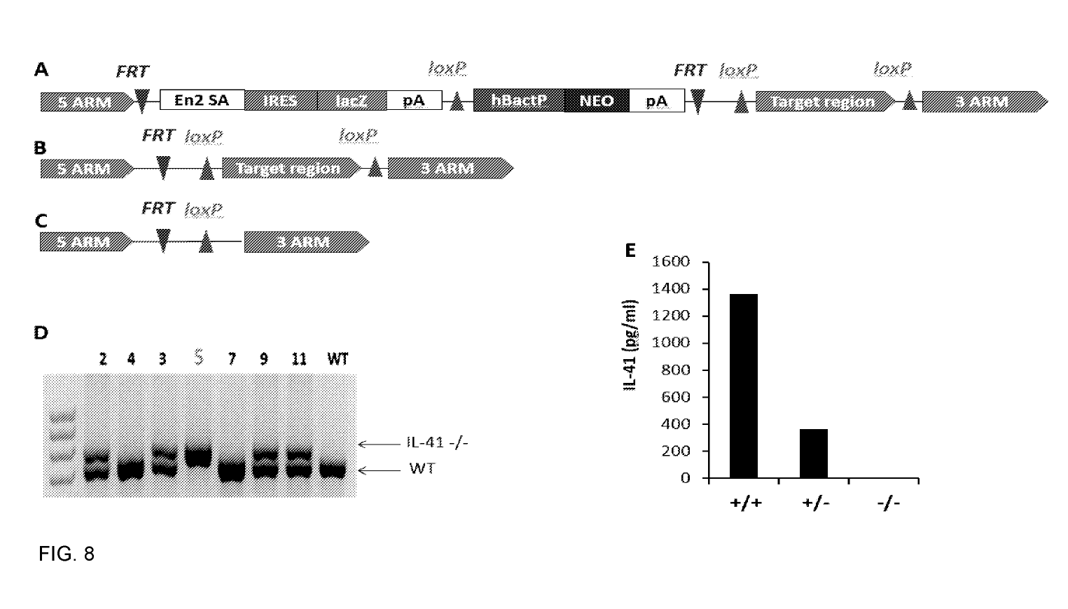

Metrnl/Il-41−^{/-} mice exhibit immune system abnormalities, including altered cytokine and chemokine expression and altered inflammatory responses. We have successfully produced a Metrnl/Il-41−^{/-} mouse (FIG. 8). Metrnl/IL-41 has been reported to be involved in metabolic regulation of energy expenditure, and control of glucose tolerance [2, 4]. The Metrnl/Il-41−^{/-} mouse exhibits normal development, gains weight normally, is otherwise healthy, and breeds well (unpublished observations). However, Metrnl/Il-41−^{/-} mice exhibit immune system abnormalities. In order to produce a Metrnl/IL-41−/− mouse (FIG. 8A), we obtained Mouse Embryonic Stem (ES) cells (obtained from the Knockout mouse project: KOMP) containing a selection cassette that was inserted into the mouse Metrnl locus by homologous recombination. These ES clones were microinjected into C57BL/6 blastocysts, which were transferred into pseudo-pregnant female mice. (FIG. 8B) Resulting chimeras were crossed with WT C57BL/6 mice and the pups were screened for germline transmission. MetrnlNeo/+ heterozygotes were subsequently bred with FLPeR mice to remove the neomycin resistance cassette. (FIG. 8C) The Metrnl Neo−/loxP+ mice were then bred to CRE mice to delete the loxP-flanked target region. Finally, heterozygote Metrnl+/− mice were intercrossed to successfully generate Metrnl−/− mice. The deletion of the target region was verified by PCR (FIG. 8D) and ELISA (FIG. 8E).

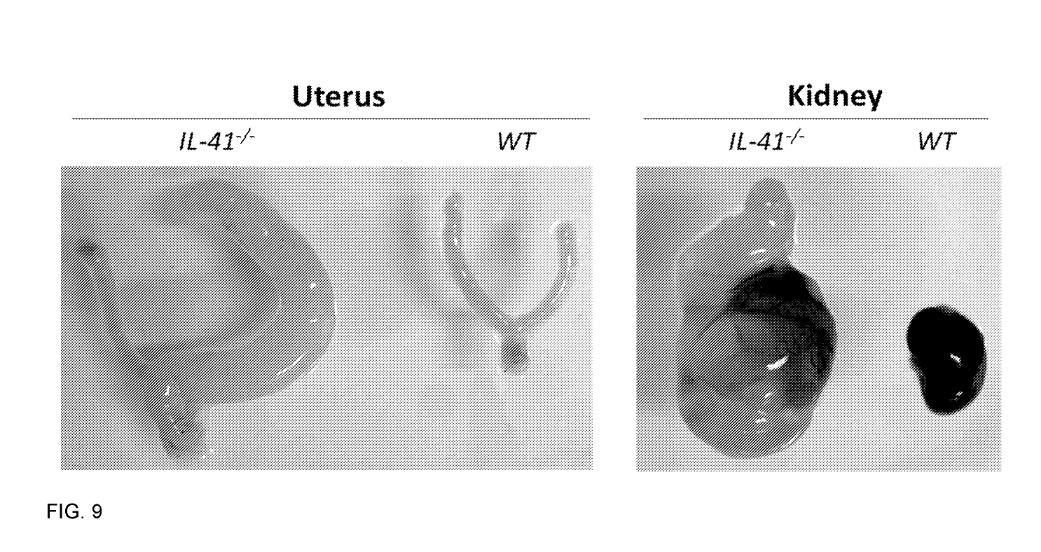

The Metrnl/Il-41−^{/-} mice can spontaneously exhibit extreme inflammatory lesions in the uterus and kidney. FIG. 9 shows inflammatory lesions in uterus (left) or kidney (right) of IL-41−/− mice. The lesions in the uterus typically showed unilateral features while the kidneys usually only affected one.

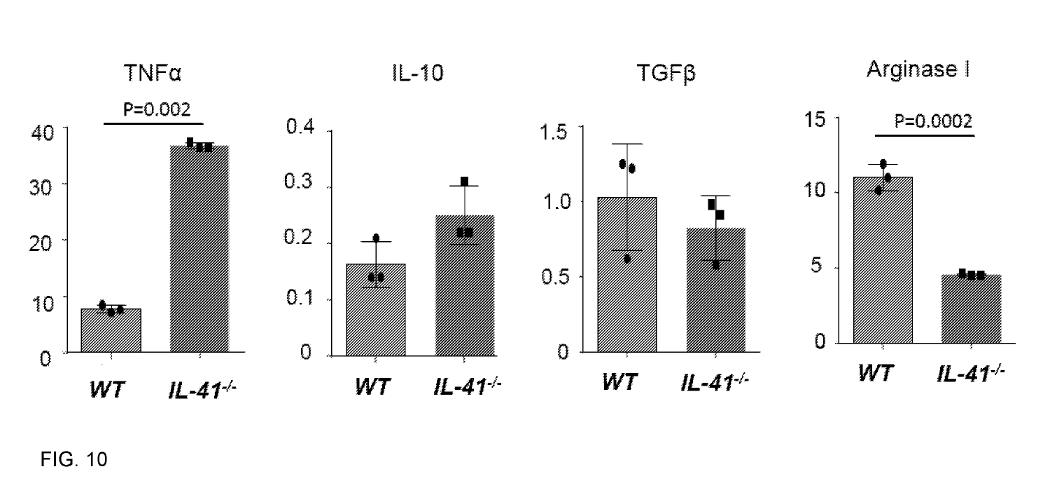

These are not seen in every animal, but appear randomly, consistent with induction due to an infectious event (e.g., bacteria, virus). These lesions are full of activated myeloid cells. These lesions likely arise from altered immune homeostasis due to the lack of expression of Metrnl/IL-41 which normally keeps pro-inflammatory mechanisms in check. These instances of gross inflammation provided an opportunity to inquire about the status of biomarkers of inflammation and to relate their status to the lack of Metrnl/IL-41. Results for the uterus sample and the kidney sample are shown in FIGS. 10 and 11, respectively. The affected tissues (uterus, kidney) were dissected and the cells from the lesions were recovered. The cells were subjected to FACS analyses, which indicated the presence of proinflammatory cells (granulocytes, macrophages), and mRNA was prepared from these cells and analyzed for several cytokines and chemokines by qPCR. The results indicate that many pro-inflammatory cytokines and chemokines were present in these lesions.

As predicted for a molecule intimately involved with inflammation, mRNA levels for proinflammatory TNFα in the uterus and of TNFα, IFNγ, CXCR2 and CCL2 in the kidney were all significantly increased, whereas Arginase I, a marker of anti-inflammatory, alternatively activated macrophages, was reduced in the uterus sample. These results are consistent with an uncontrolled inflammatory response associated with M1, or classically-activated, macrophages in the absence of Metrnl/IL-41.

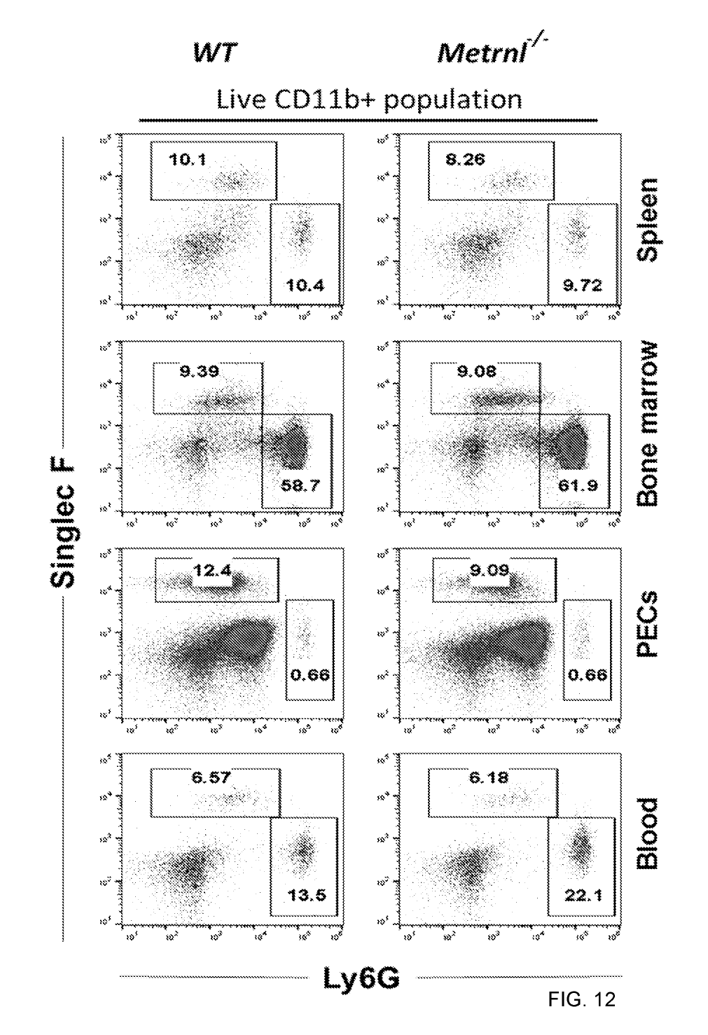

Cell populations in different body compartments of the healthy Metrnl/IL-41−/− mice were studied by flow cytometry (FIG. 12). As shown in FIG. 12. IL-41−/− mice have increased numbers of neutrophils in the blood. Representative plots and frequency of neutrophil and eosinophil populations (gated on CD11b⁺) in different organs of WT and IL-41^{-/-} mice are shown.

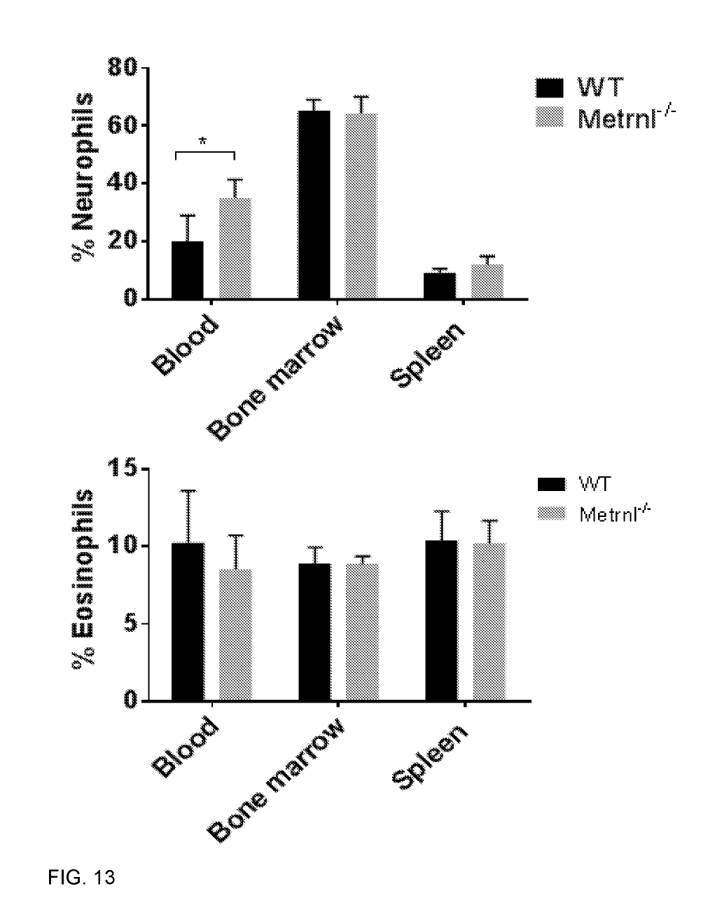

We observed a significant neutrophilia, as evidenced by Ly6G expression, in the blood of the Metrnl/IL-41−/− mice, but no significant changes were observed for eosinophils (Siglec F⁺ cells) in any of the compartments (FIG. 13). As shown in FIG. 13, IL-41−/− mice have neutrophilia in the blood. Data from 5 to 7 mice are shown. Student's t-test was performed on the indicated groups. *P<0.05. Eosinophil and neutrophil populations were verified using Amnis Imagestream and were not affected in blood, bone marrow or spleen.

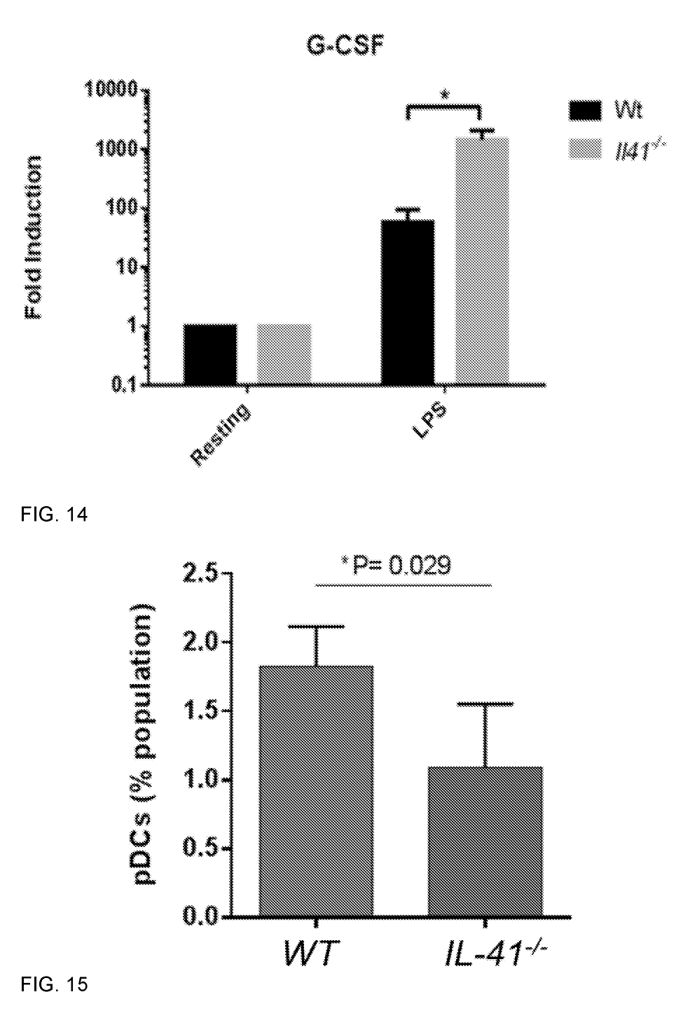

To explain the neutrophilia in the Metrnl/IL-41−/− mice, we studied the capacity of activated Metrnl/IL-41−/− peritoneal macrophages to produce G-CSF. G-CSF is responsible for driving neutrophil production. FIG. 14 shows that LPS-activated macrophages (10 ng/24 h) from Metrnl/IL-41−/− mice produce significantly more G-CSF as compared to the WT control mice. This is consistent with the observed neutrophilia. Results are representative of at least 2 separate experiments.

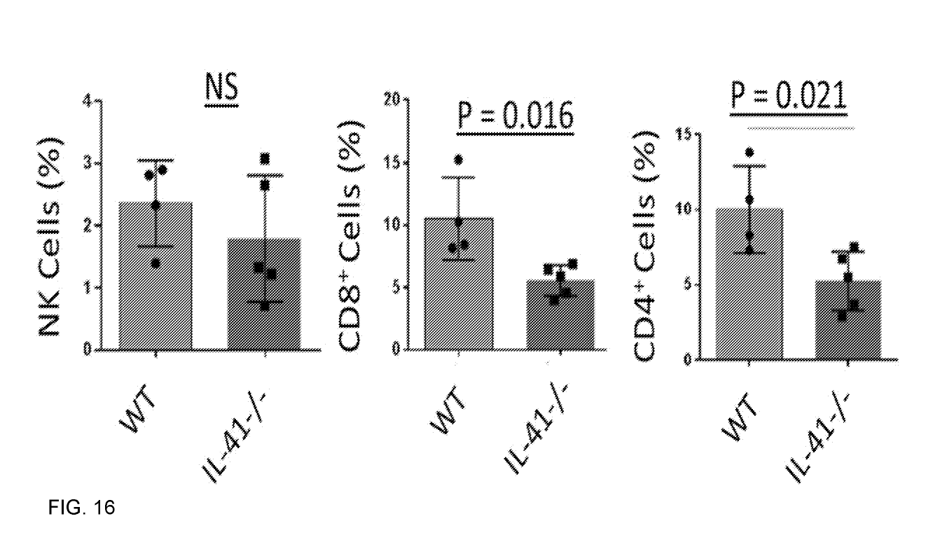

The numbers of plasmacytoid dendritic cells (pDC) were reduced in the spleens of the Metrnl/IL-41−/− mice (FIG. 15) while the CD11b+ dendritic cell population (DCs) were unaffected (data not shown). For the results described in FIGS. 15 and 16, spleens from 5-7 mice (IL-41−/− or controls) were phenotyped for plasmacytoid dendritic cells (Ly6C+) as well as for CD8+ T cells or CD11b+ dendritic cells by FACS. Only the plasmacytoid dendritic cells were affected. And, the numbers of CD4⁺ and CD8⁺ T cells in the spleen were also significantly reduced in the Metrnl/IL-41−/− mice, while the numbers of NK cells were not affected (FIG. 16).

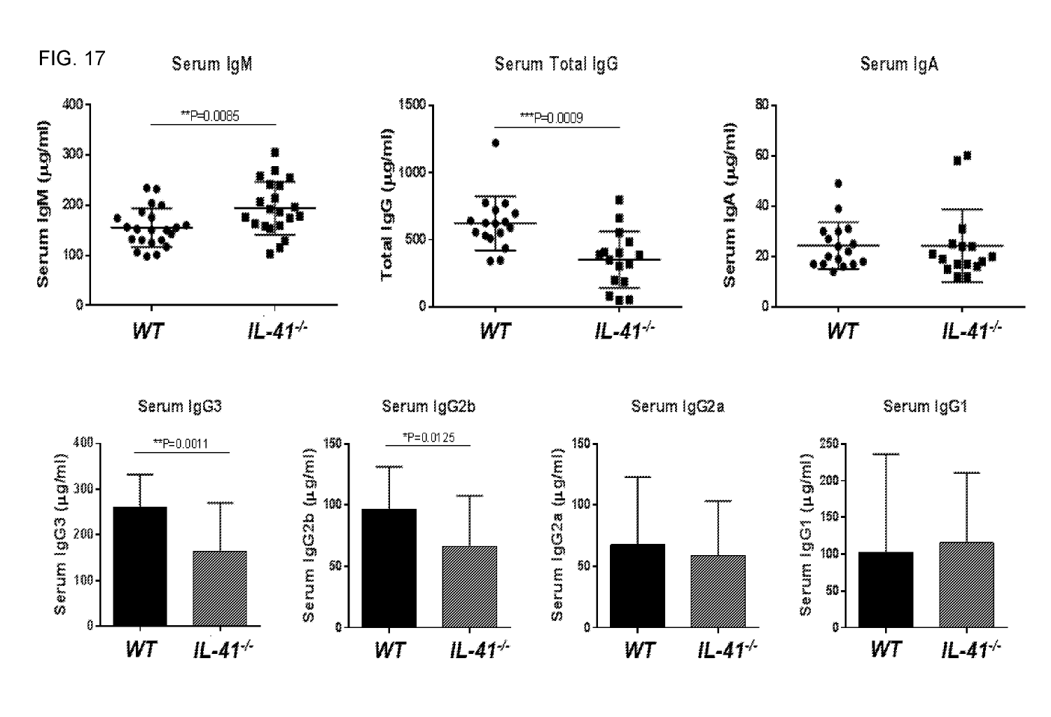

Metrnl/Il-41−^{/-} mice have significantly lower levels of serum IgG. Further analyses revealed that this is due to strongly reduced levels of IgG2b and IgG3 (FIG. 17).

Serum from IL-41−/− mice were tested by ELISA for IgM, IgG, and IgA. Having detected lower levels of IgG, we repeated the experiment measuring IgG3, IgG2b , IgG2a and IgG1 levels by ELISA. The data indicate that IL-41−/− mice have significantly lower levels of IgG3, and IgG2b.

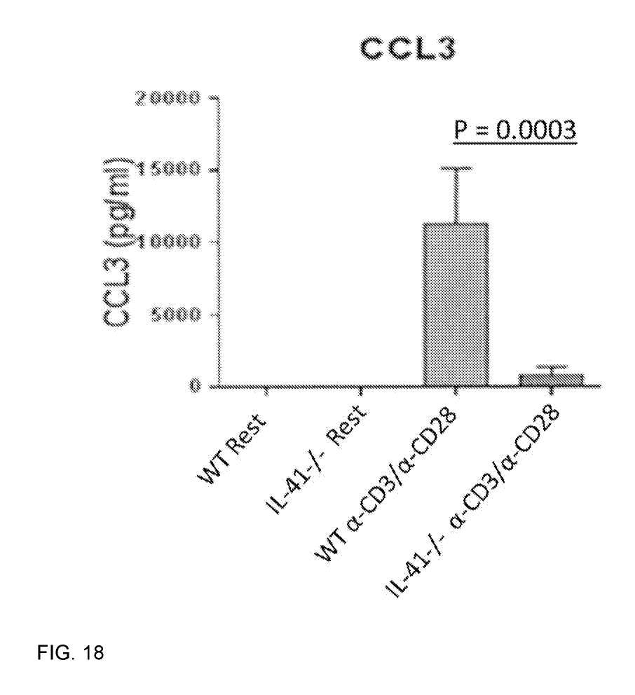

Metrnl/IL-41 has been shown to induce the expression of Peroxisome proliferator-activated receptor γ (PPARγ) in adipocytes. Furthermore, data from the BIGE database indicates that PPARγ is expressed in adipose tissue and also leukocytes, including lymphocytes (not shown). Importantly, PPARγ has been shown to be expressed in T cells during sepsis. This expression pattern fits with the reported effects of Metrnl/IL-41, which include metabolic and immunoregulatory/inflammatory effects. Importantly, PPARγ is known to be involved in anti-inflammatory mechanisms through its ability to modulate cytokine production. We therefore predicted that Metrnl/Il-41−^{/-} leukocytes would exhibit altered cytokine and chemokine production. We have observed that the capacity of activated Metrnl/Il-41−^{/-} T cells to produce CCL3 is strongly inhibited (FIG. 18). CCL3 is a chemokine produced during early phase of primary infections and recruits T and NK cells. Spleen T cells from WT or IL41−/− mice were stimulated with anti-CD3/CD28 in solid phase for 24 h and CCL3 was measured by ELISA.

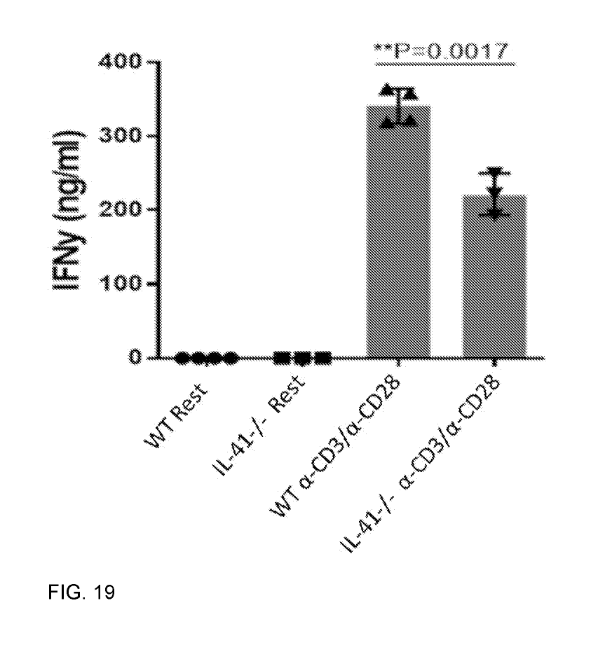

In addition, the T cells from IL41−/− mice also produce significantly lower levels of IFNγ, (FIG. 19) suggesting that the differentiation pathway towards Th1 cells may be abnormal. Spleen T cells from WT or IL-41−/− mice were stimulated with anti-CD3/CD28 in solid phase for 24 h and IFNγ was measured by ELISA.

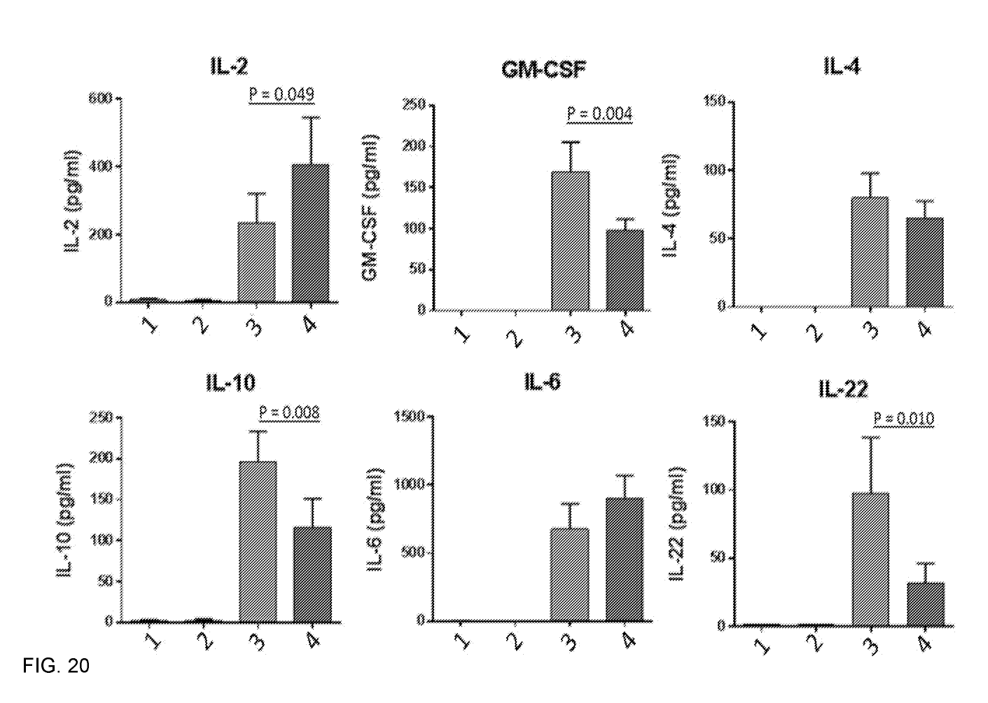

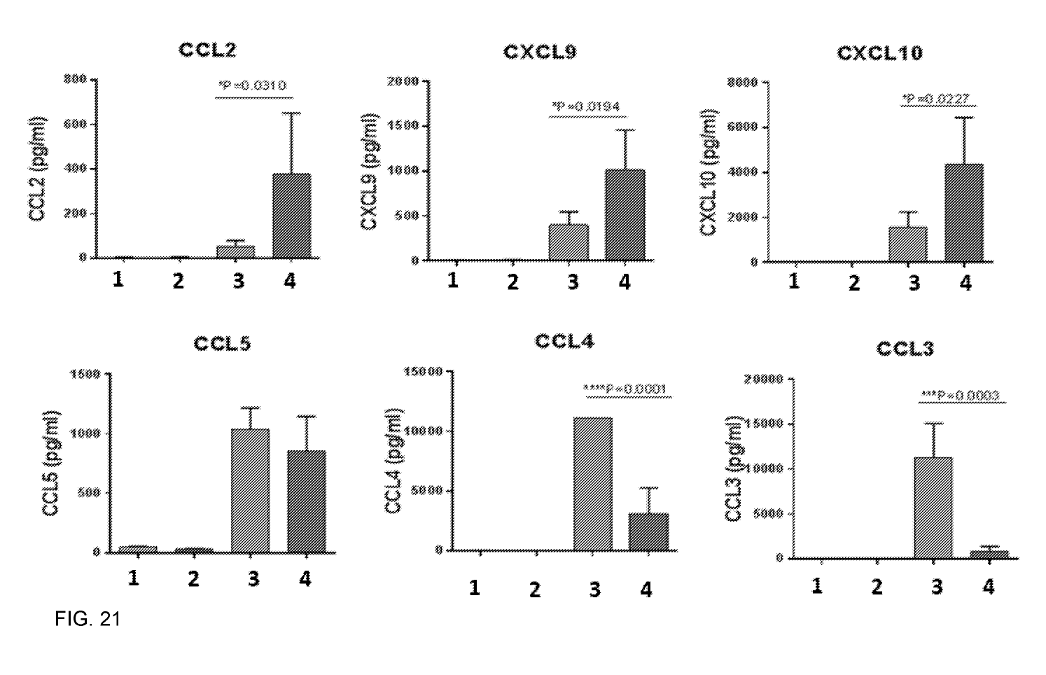

Additional immunomodulatory cytokine and chemokine production was measured in anti-CD3 and anti-CD28 activated T lymphocytes within the splenocyte population obtained from Metrnl/IL-41−/− mice. Assays were performed by multiplex ELISA techniques and confirmed by traditional ELISA. Levels of various cytokines produced by splenocytes from WT or Metrnl/IL41−/− mice following stimulation with anti-CD3 and anti-CD28 in solid phase for 24 h. Supernatants were collected at 24 h and tested by ELISA for the corresponding cytokines. FIG. 20 shows statistically significant increases of IL-2 production and decreases of IL-10, IL-22 and GM-CSF by anti-CD3/CD28 splenocytes from solid-phase activated T cells from Metrnl/IL-41−/− mice. Similarly, significant changes in chemokine production were seen by the activated T cells obtained from the Metrnl/IL-41−/− mice (FIG. 21). These results are all consistent with an altered immune response regulation capacity that could explain the gross inflammation observed in the Metrnl/IL-41−/− mice.

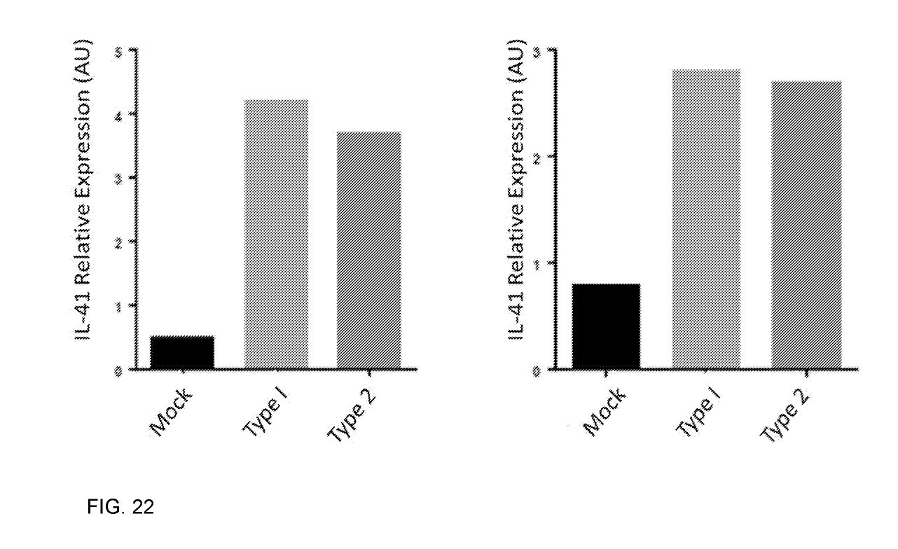

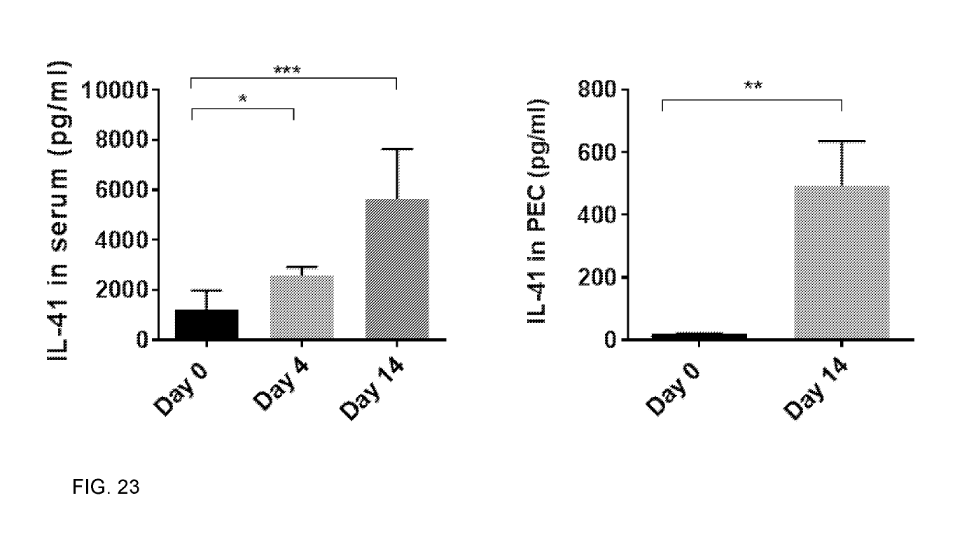

Another model of inflammatory challenge was evaluated in WT and Metrnl/IL-41−/− mice to study the association of Metrnl/IL-41 with inflammation. Cytokines (especially IFNγ) are important in resistance to certain pathogens, suggesting that the dysregulation in cytokine production would render Metrnl/Il-41−^{/-} mice more susceptible to pathogen. It is therefore expected that Metrnl/Il-41−^{/-} mice will be more susceptible to infection for example with Toxoplasma gondii [20] and other intracellular parasites. Following infection by the Toxoplasma gondii oocyst in the WT mouse, a generalized inflammatory response is triggered that leads to extreme cytokine production in the host, followed by death due to the cytokine storm, including IL-41, generated as the host attempts to eradicate the parasite. The following figure shows that Metrnl/IL-41 is produced by THP-1 macrophage cells infected with either of two different strains of Toxoplasma gondii (FIG. 22). This indicates that Metrnl/IL-41 is normally produced during Toxoplasma infection. The following figure shows that IL-41 is produced constantly for at least 14 days in the WT mouse in response to infection (FIG. 23). IL-41 levels (measured by ELISA) in mice serum of mice infected with T. gondii (left) or in supernatants of peritoneal exudate cells infected with T. gondii for 24 h (right).

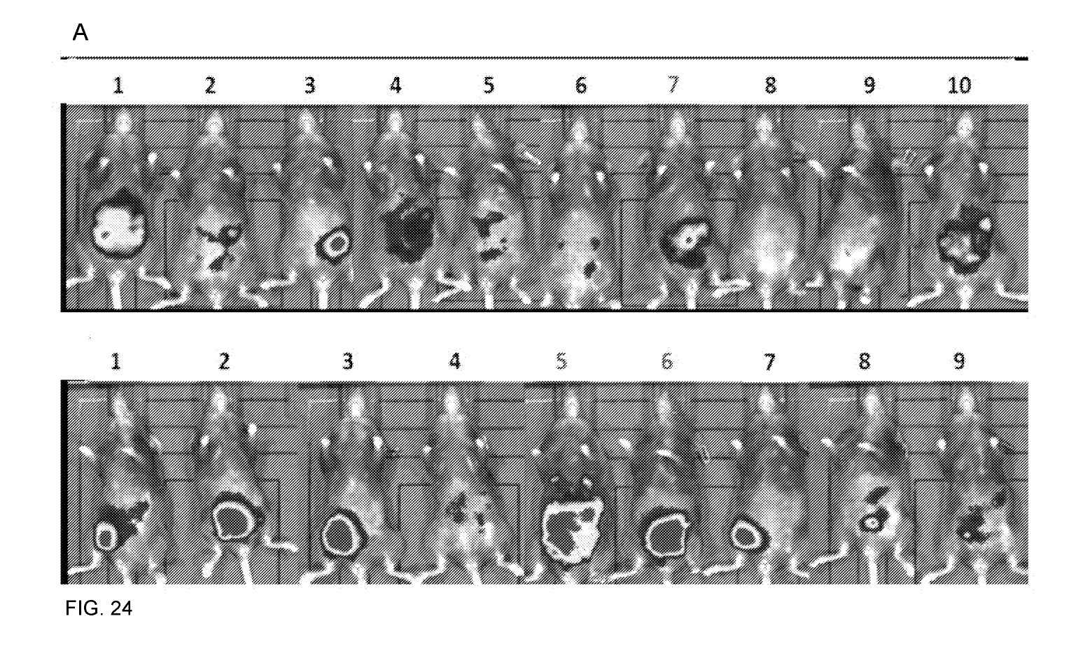

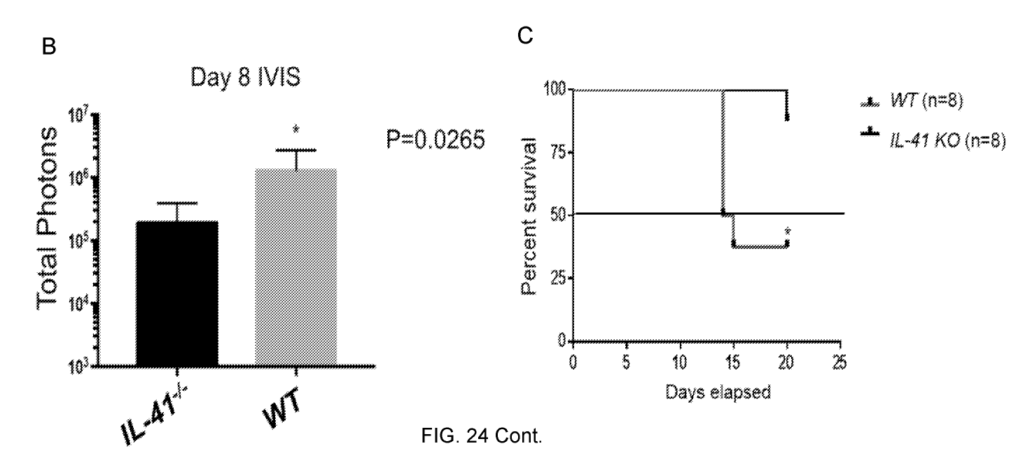

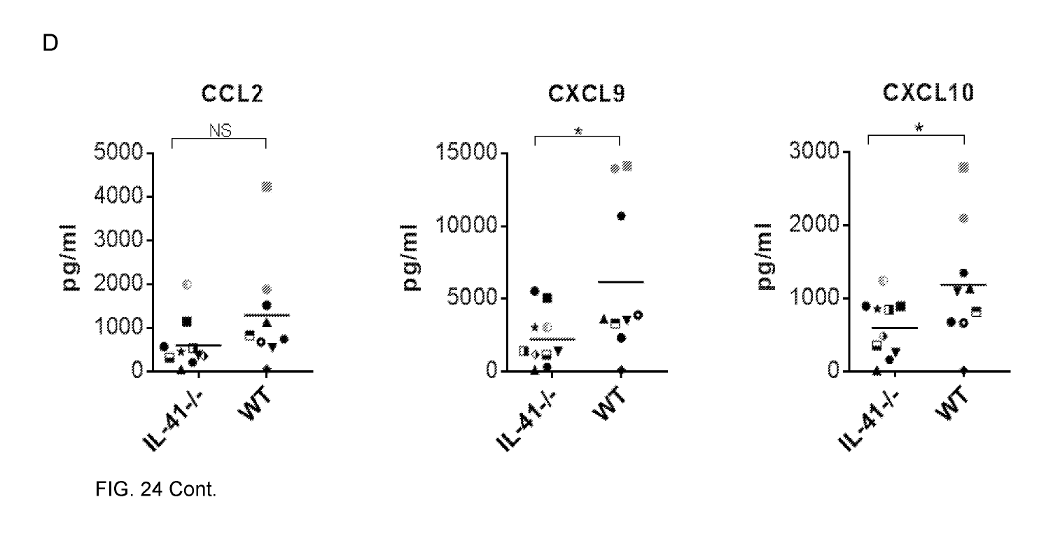

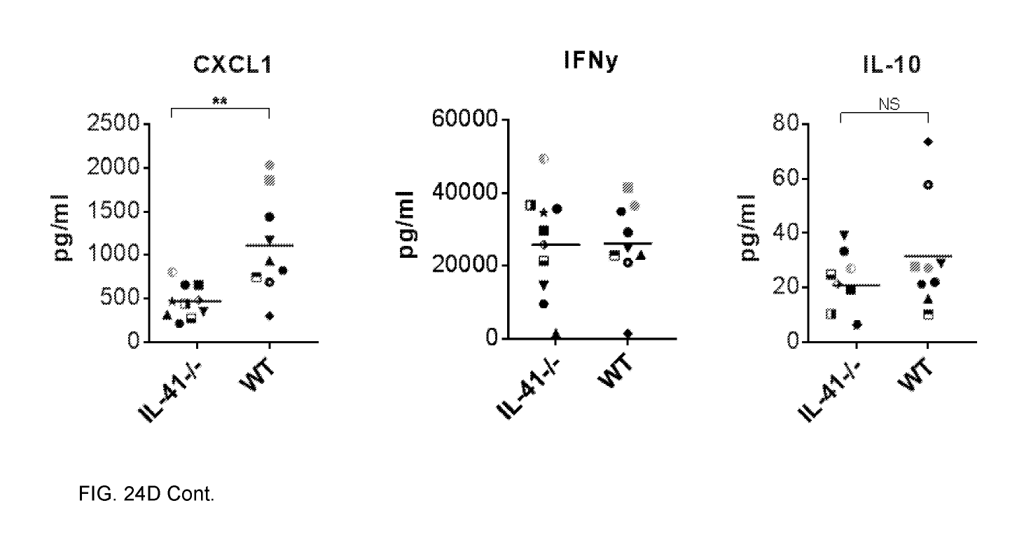

When the WT and Metrnl/IL-41−/− mice are treated with T. Gondii and the degree of infection monitored by whole body imaging, the WT mice exhibited a greater parasite load and began to die from the infection sooner as compared to the Metrnl/IL-41−/− mice (FIG. 24). IL-41−/− mice are more resistant to T. gondii infection than WT mice (FIG. 24A). Nine WT and ten IL-41−/− mice were infected i.p. with 200 T. gondii tachyzoites expressing luciferase. On day 8 p.i. parasite burden was measured using a bioluminescence imaging (BLI) system. (FIG. 24B) BLI quantitation of the mice shown in 4A indicates that WT mice had significantly higher photon signal from T. gondii infection than Il-41−/− mice (*p<0.0265). (FIG. 24C) In a second experiment, WT and IL-41−/− mice were infected i.p. with 500 T. gondii tachyzoites and the mouse survival was monitored until day 20 p.i. *p<0.05.

Measurement of serum cytokine and chemokine levels in the two populations showed significant reductions of IL-10, CCL2, CXCL9, CXCL10 and CXCL1 in the Metrnl/IL-41−/− mice as compared to control consistent with a reduced cytokine storm (FIG. 24D). On day 4 post injection (p.i.) with 200 T. gondii tachyzoites, levels of various cytokines and chemokines from the experiment shown in FIG. 24 were measured using Legendplex (BioLegend). Day 4 levels of measured cytokines/chemokines were then correlated with a survival outcome at day 14 p.i. (mice that survived until 14 days p.i. are marked in black and mice that died are marked in red) (*:p<0.05; **:p<0.02). The data indicate that the mice that died had the highest levels of cytokines/chemokines measured (except IL-10). Toxoplasma susceptibility depends on the production of pro-inflammatory cytokines during the infection. Mice die of cytokine storm due to the cytokine production induced by the parasite (Mordue, D. G., F. Monroy, M. La Regina, C. A. Dinarello, and L. D. Sibley. 2001. Acute toxoplasmosis leads to lethal overproduction of Th1 cytokines. J. Immunol 167: 4574-4584, incorporated by reference herein). Therefore, these results indicate that Metrnl/IL-41 can influence the production of certain cytokines in vivo and its absence in the Metrnl/IL-41−/− mouse results in a deficiency of cytokine production during the infection, which in turn protects the mice from death.

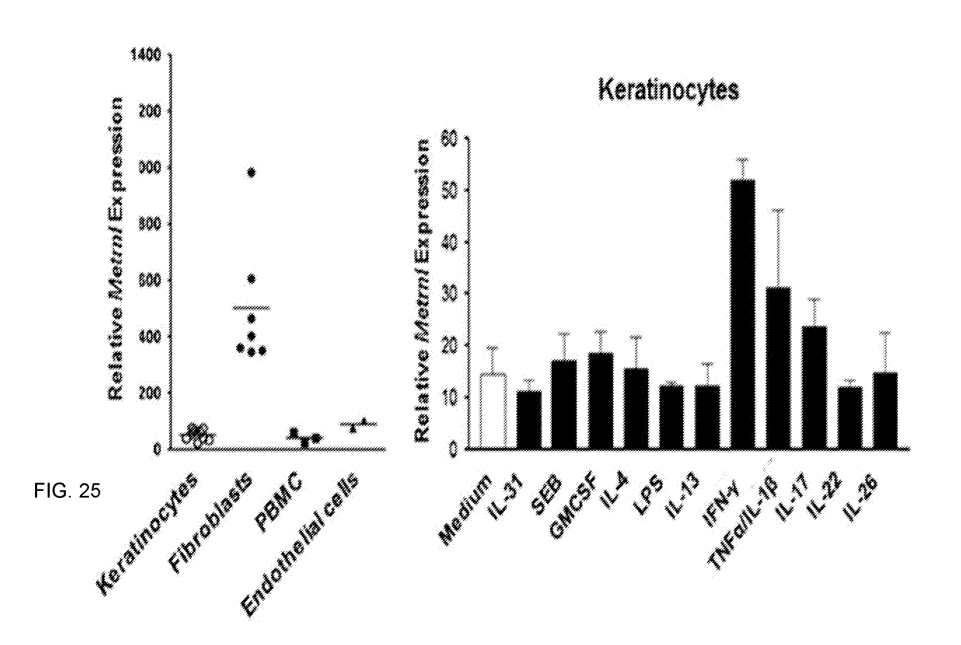

Metrnl/IL-41 expression is associated with human diseases. We have explored a possible role for Metrnl/IL-41 in human diseases. We have shown that in the skin, Metrnl/IL-41 is expressed by dermal fibroblasts and in endothelial cells, and can also be upregulated by IFNγ in cultured human keratinocytes (FIG. 25).

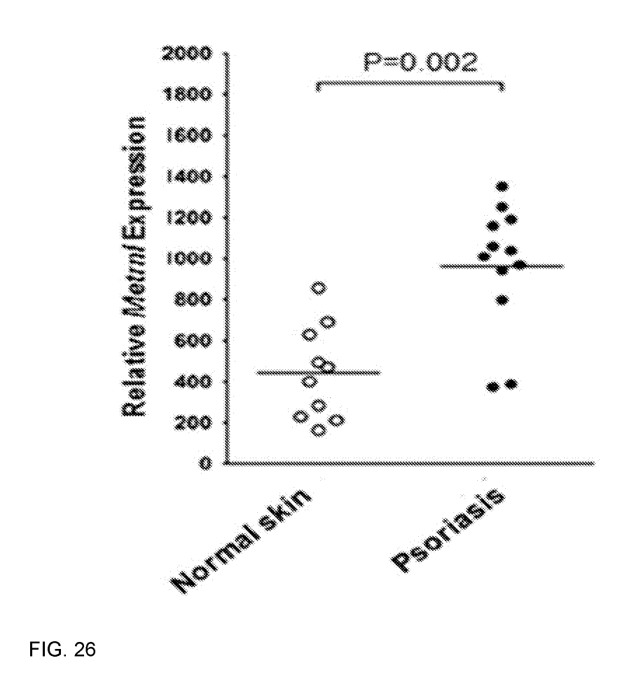

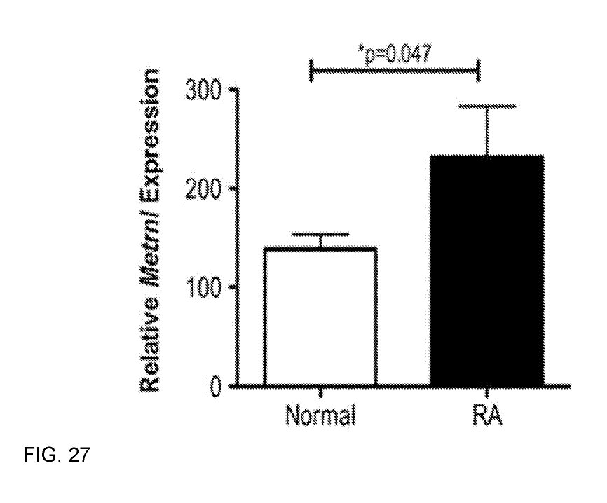

We have analyzed METRNL/IL-41 expression in various human skin diseases and observed that METRNL/IL-41 is strongly over-expressed in human psoriatic skin (FIG. 26), It is also upregulated in synovial membranes of human rheumatoid arthritis (FIG. 27). Biopsy samples from human lesions corresponding to these diseases were obtained and cDNA was produced. These samples were used for qPCR for Metrnl/IL-41 (Systematic identification and characterization of novel human skin-associated genes encoding membrane and secreted proteins. Gerber P A, Hevezi P, Buhren B A, Martinez C, Schrumpf H, Gasis M, Grether-Beck S, Krutmann J, Homey B, Zlotnik A. PLoS One. 2013 Jun. 20; 8(6):e63949, incorporated by reference herein). Also, we detected METRNL/IL-41 overexpression in specific human skin diseases, particularly in atopic dermatitis, psoriasis, prurigo nodularis and actinic keratosis (Table 2). Biopsy samples from human lesions corresponding to these diseases were obtained and cDNA was produced. These samples were used for qPCR for Metrnl/IL-41. (Gerber P A, Hevezi P, Buhren B A, Martinez C, Schrumpf H, Gasis M, Grether-Beck S, Krutmann J, Homey B, Zlotnik A. PLoS One. 2013 Jun. 20; 8(6):e63949); Soto H, Hevezi P, Roth R B, Pahuja A, Alleva D, Acosta H M, Martinez C, Ortega A, Lopez A, Araiza-Casillas R, Zlotnik A. Gene array analysis comparison between rat collagen-induced arthritis and human rheumatoid arthritis. Scand J Immunol. 2008 July; 68(1):43-57, incorporated by reference herein).

TABLE 2
Expression of Metrnl in patients with skin diseases

| Diseases | Patients^{a} | | Healthy controls^{a} | P value |
|---|---|---|---|---|
| Inflammatory | | | | |
| Atopic dermatitis | 685.3 | (523.6-744.2) | 436.3 (230.6-630.1) | 0.034 |
| Psoriasis | 1024 | (869.8-1176.0) | 436.3 (230.6-630.1) | 0.002 |
| Prurigo nodularis | 646.9 | (515.4-744.2) | 436.3 (230.6-630.1) | 0.045 |
| Lupus erythematous | 414.8 | (342.4-564) | 436.3 (230.6-630.1) | 0.755 |
| Lichen planus | 499.7 | (453.4-586.7) | 436.3 (230.6-630.1) | 0.427 |
| Neoplastic | | | | |
| Actinic keratosis | 739.7 | (608.7-1167.8) | 436.3 (230.6-630.1) | 0.017 |
| Basal cell carcinoma | 411.5 | (189.5-624.5) | 436.3 (230.6-630.1) | 0.784 |
| Squamous cell carcinoma | 328.5 | (268.4-613.9) | 436.3 (230.6-630.1) | 0.949 |

^{a}Median; interquartile range in parentheses. Healthy controls N = 10; patients, a minimum N = 6 was used.

These observations and the preceding data regarding an association with inflammation suggest that METRNL/IL-41 may be involved in the pathogenesis of certain human autoimmune or inflammatory diseases, particularly in skin diseases or at barrier sites.

Another finding is that Metrnl/IL-41 induces the expression of CCL2/MCP-1 in activated T cells, or CXCL1, CCL2, CXCL10, CCL4 CXCL5, CCL22 or CXCL13 expression by macrophages. This can be the basis of a bioassay that involves incubating spleen or lymph node T cells (T cells can be purified from single cell suspensions from these organs with kits such as those obtained from Miltenyi Biotech or MojoSort from BioLegend) with a mitogen (concanavalin A) or anti-CD3 and anti-CD28 antibodies in solid phase in the presence or absence of Metrnl/IL-41. After 24, 48 or 96 hours, the supernatant can be tested by ELISA for the presence of CCL2/MCP-1. There are commercially available ELISAs for CCL2 from Biotechne (Minneapolis, Minn.) or BioLegend (San Diego, Calif.). Biologically active IL-41 will increase the amount of CCL2 produced by the T cells. Alternatively, the biological activity assay could be performed using macrophages, which can be obtained by washing the peritoneal cavity of mice, by washing the cavity with 4 ml of saline solution. The cells are plated in 24 well plates for 1 hour at 37° C. and the non-adherent cells are discarded. The adherent cells (mostly macrophages) will be cultured in the presence or absence of Metrnl/IL-41 and 24, 48 or 96 h later, the supernatants are removed and assayed for the presence of CXCL1, CCL2, CXCL10, CCL4, CXCL5, CCL22 or CXCL13 by ELISA. Biologically active IL-41 increases the production of these chemokines. Alternatively, the latter assay can be performed in the presence of 10 ng/ml of lipopolysaccharide (LPS) from E. coli 0111:B4 (Cat No. L-4391, Sigma). Again, addition of IL-41 increases the production of the chemokines listed above by macrophages. The increase in these chemokines produced by macrophages or related cells (epithelial, endothelial) can be used to detect bioactive IL-41.

### EXAMPLE 2

### Methods and Materials

Metrnl protein levels were measured by ELISA product obtained commercially from R&D systems (Minneapolis, Minn., USA; see the World Wide Web at rndsystems.com/products/mouse-meteorin-like-metrnl-duoset-elisa_dy6679). Briefly, the product is a solid phase sandwich ELISA product with a capture antibody and a detection antibody. The latter is a rat IgG2b clone #829535 from R&D systems (Cat #MAB66791). A reference recombinant protein Meteorin-like is used to generate a standard curve.

Other cytokines can be measured and detected using standard ELISA and radioimmunoassay methods. For example, assays are commercially available from R&D systems (Minneapolis, Minn., USA), BioLegend (San Diego, Calif., USA) or ThermoFisher/Ebioscience (Carlsbad, Calif., USA).

### REFERENCES

The following publications are incorporated by reference herein in their entireties:
Ushach, I, Burkhardt, A M, Martinez, C. Hevezi, P A, Buhren, B A, Schrumpf, H., Valle-Rios, R., Vazquez, M I, Homey, B., and and Zlotnik. A. 2015. Meteorin-like is a cytokine associated with barrier tissues and alternatively activated macrophages. Clin Immunol. 2015 February; 156(2):119-27. doi: 10.1016/j.clim.2014.11.006.
Rao R R, Long J Z, White J P, Svensson K J, Lou J, Lokurkar I, Jedrychowski M P, Ruas J L, Wrann C D, Lo J C, Camera D M, Lachey J, Gygi S, Seehra J, Hawley J A, Spiegelman B M. Meteorin-like is a hormone that regulates immune-adipose interactions to increase beige fat thermogenesis. 2014. Cell. 157: 1279-91 doi:10.1016/j.cell.2014.03.065.
Gene expression analyses reveal molecular relationships among 20 regions of the human CNS. Roth R B, Hevezi P, Lee J, Willhite D, Lechner S M, Foster A C, Zlotnik A. Neurogenetics. 2006 May; 7(2):67-80.
Effects of RNA degradation on gene expression analysis of human postmortem tissues. Lee J, Hever A, Willhite D, Zlotnik A, Hevezi P. FASEB J. 2005 August; 19(10):1356-8.
P. A. Gerber, P. Hevezi, B. A. Buhren, C. Martinez, H. Schrumpf, M. Gasis, S. Grether-Beck, J. Krutmann, B. Homey, A. Zlotnik, Systematic identification and characterization of novel human skin-associated genes encoding membrane and secreted proteins, PLoS One 8 (2013) e63949.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of modulating a condition selected from cytokine release syndrome, systemic immune response syndrome, cytokine storm, or a combination thereof, in a subject in need of such modulating, comprising:
administering to the subject an effective amount of Meteorin-like (Metrnl/IL-41), a bioactive fragment of Metrnl/IL-41, a genetically or chemically modified Metrnl/IL-41, or a combination thereof, or administering one or more anti-Metrnl/IL-41 antibodies.

2. The method of claim 1, wherein the modulating comprises reducing the severity of the condition.

3. The method of claim 1, the wherein the subject has sepsis.

4. The method of claim 1, wherein an effective amount of Meteorin-like (Metrnl/IL-41), a bioactive fragment of Metrnl/IL-41, a genetically or chemically modified Metrnl/IL-41, or a combination thereof, is administered.

5. There method of claim 1, wherein an effective amount of one or more anti-Metrnl/IL-41 antibodies is administered.

6. The method of claim 5, wherein the one or more anti-Metrnl/IL-41 antibodies are Metrnl/IL-41 neutralizing antibodies.

7. The method of claim 1, wherein after the administering, the subject is monitored for one or more symptoms of the condition.

8. The method of claim 7, wherein the symptoms include serum levels of interleukin-6, interleukin-1, or TNFα, or a combination thereof.

9. The method of claim 1, wherein the cytokine storm is a result of one or more of: bacteremia, bacterial sepsis, cellulitis, cholecystitis, community-acquired pneumonia, diabetic foot infection, erysipelas, HIV (acute retroviral syndrome), infective endocarditis, influenza, intra-abdominal infections, meningitis, necrotizing fasciitis, nosocomial pneumonia, pelvic inflammatory disease, prostatitis, pseudomembranous colitis (Clostridium difficile), pyelonephritis, septic arthritis, or systemic inflammatory response syndrome (SIRS), toxic shock syndrome, urinary tract infection, viral syndrome, acute mesenteric ischemia, acute respiratory distress syndrome, alcohol withdrawal, burns, cirrhosis, connective tissue disease, deep venous thrombosis, dehydration, DKA, drug overdose, drug reaction, electrical injuries, erythema multiforme, gastrointestinal bleeding, gout, graft versus host disease, hemorrhagic shock, intestinal perforation, malignancy, myocardial infarction, pancreatitis, peripheral ischemia, pulmonary embolism, toxic epidermal necrolysis, transfusion reactions or trauma.

10. The method of claim 1, wherein the cytokine release syndrome is a result of one or more of: sepsis, CAR-T cell toxicity, or diseases caused by superantigens such as, for example, staphylococcal enterotoxin B, toxin shock toxin.

11. The method of claim 1, wherein the administering is by intravenous administration.

12. A method of treating rheumatoid arthritis or psoriasis in a subject in need thereof, comprising administering an effective amount of Metrnl/IL-41, a bioactive fragment of Metrnl/IL-41, a genetically or chemically modified Metrnl/IL-41, or a combination thereof, to the subject.


SEQUENCE LISTING (5 sequences)

SEQ ID NO: 1 - protein, 293 aa, Homo sapiens
MGFPAAALLCALCCGLLAPAARAGYSEERCSWRGSGLTQEPGSVGQLALACAEGAVEWLYPAGALRLTLGGPDPRARPGIACLRPVRPFAGAQVFAERAGGALELLLAEGPGPAGGRCVRWGPRERRALFLQATPHQDISRRVAAFRFELREDGRPELPPQAHGLGVDGACRPCSDAELLLAACTSDFVIHGIIHGVTHDVELQESVITVVAARVLRQTPPLFQAGRSGDQGLTSIRTPLRCGVHPGPGTFLFMGWSRFGEARLGCAPRFQEFRRAYEAARAAHLHPCEVALH

SEQ ID NO: 2 - protein, 311 aa, homo sapiens
MRGAARAAWGRAGQPWPRPPAPGPPPPPLPLLLLLLAGLLGGAGAQYSSDRCSWKGSGLTHEAHRKEVEQVYLRCAAGAVEWMYPTGALIVNLRPNTFSPARHLTVCIRSFTDSSGANIYLEKTGELRLLVPDGDGRPGRVQCFGLEQGGLFVEATPQQDIGRRTTGFQYELVRRHRASDLHELSAPCRPCSDTEVLLAVCTSDFAVRGSIQQVTHEPERQDSAIHLRVSRLYRQKSRVFEPVPEGDGHWQGRVRTLLECGVRPGHGDFLFTGHMHFGEARLGCAPRFKDFQRMYRDAQERGLNPCEVGTD

SEQ ID NO: 3 - protein, 311 aa, Homo sapiens; identical to SEQ ID NO: 2
  SIGNAL (1)..(45)

SEQ ID NO: 4 - protein, 133 aa, Homo sapiens
QYSSDRCSWKGSGLTHEAHRKEVEQVYLRCAAGAVEWMYPTGALIVNLRPNTFSPARHLTVCIRSFTDSSGANIYLEKTGELRLLVPDGDGRPGRVQCFGLEQGGLFVEATPQQDIGRRTTGFQYELVRRHRA

SEQ ID NO: 5 - protein, 133 aa, Homo sapiens
SDLHELSAPCRPCSDTEVLLAVCTSDFAVRGSIQQVTHEPERQDSAIHLRVSRLYRQKSRVFEPVPEGDGHWQGRVRTLLECGVRPGHGDFLFTGHMHFGEARLGCAPRFKDFQRMYRDAQERGLNPCEVGTD